(19) United States
(12) Patent Application Publication
(10) Pub. No.: US 2026/0226149 A1
(43) Pub. Date: Aug. 6, 2026

(54) TREATMENT OF CHRONIC RHINOSINUSITIS WITH ANTI-TSLP ANTIBODY

(71) Applicant: MEDIMMUNE LLC, Gaithersburg, MD (US)

(72) Inventor: Janet Griffiths, Gaithersburg, MD (US)

(21) Appl. No.: 19/152,041

(22) PCT Filed: Feb. 2, 2024
(86) PCT No.: PCT/US2024/014361

§ 371 Date: Jul. 30, 2025

Related U.S. Application Data
(60) Provisional application No. 63/483,000, filed on Feb. 2, 2023.
(60) Provisional application No. 63/503,087, filed on May 18, 2023.

(51) Int. Cl. C07K 16/24; A61K 39/00; A61P 11/02

(52) U.S. Cl. CPC C07K 16/244; A61P 11/02; A61K 2039/505; A61K 2039/54; A61K 2039/545

(57) ABSTRACT

The present disclosure, relates, in general, to methods of treating chronic rhinosinusitis, with or without nasal polyps, using an antibody specific for thymic stromal lymphopoietin (TSLP).

### CROSS REFERENCE TO RELATED APPLICATIONS

The present application claims the priority benefit of U.S. Provisional Patent Application No. 63/483,000, filed Feb. 2, 2023, and U.S. Provisional Patent Application No. 63/503,087, filed May 18, 2023, herein incorporated by reference in their entireties.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “58714_SeqListing.xml”, which was created on Jan. 23, 2024, and is 15,609 bytes in size. The subject matter of the Sequence Listing is incorporated herein in its entirety by reference.

### FIELD OF THE DISCLOSURE

The present disclosure relates, in general, to methods of treating chronic rhinosinusitis, with or without nasal polyps, using an antibody specific for thymic stromal lymphopoietin (TSLP).

### BACKGROUND

Chronic rhinosinusitis with nasal polyps (CRSwNP) is a chronic heterogenous inflammatory condition of the nasal and paranasal mucosa (Fokkens et al 2020). The underlying pathogenesis of the disease is currently undefined but in recent years type 2 (T2) driven inflammation has been shown to be a dominant process in CRSwNP and therapeutics targeting T2 inflammation have demonstrated efficacy in CRSwNP (Gevaert et al 2011, Bachert et al 2016, Bachert et al 2017, Bachert et al 2019, Gevaert et al 2020). CRSwNP is strongly linked with asthma by epidemiological, pathophysiological, and clinical evidence, supporting an “united airway” concept to chronic inflammatory diseases of the upper and lower airways. Both diseases are comorbid conditions (Håkansson K et al 2015).

Compared to non-asthmatic patients, asthmatic patients have a higher prevalence of CRS and CRSwNP and greater severity of rhinosinusitis (Jarvis D et al 2012, Seybt M W et al 2007, Pearlman A N et al, 2009, Tint D et al 2016, Settipane G A, 1977). Furthermore, asthmatic subjects with CRSwNP may have more poorly controlled asthma, increased airway obstruction, and greater lower airway inflammation than those without CRSwNP (Bilodeau L et al 2010). Co-existing asthma in CRSwNP patients is associated with lower quality of life (Alobid I et al 2005, Ehnhage A et al 2009). Mechanistically, both NP and severe eosinophilic asthma are considered Type-2 (T2) driven diseases and the inflammatory profile is similar between the upper airway in NP and the lower airway in asthma (Fokkens et al 2020, Chaaban M R et al 2013). Like asthma, the role of eosinophils is considered important to the pathology of NP in a high proportion of patients.

Nasal polyposis represents an area of significant unmet medical need, especially for a subset of patients with NP who have exhausted current treatment options, which include medical interventions (intranasal corticosteroids (INCS), systemic corticosteroids (SCS), and antibiotics) and surgical interventions. Patients and healthcare professionals both point to a greater need for new therapies that reduce the need for repeated courses of SCS or surgery for all patients with NP. Surgery to remove polyps remains a consideration in patients whose symptoms are not controlled with corticosteroids, but rates of recurrence are high (approximately 40%, 12 months after surgery) and repeated surgical interventions are often needed. Although INCS are continued after surgery, a proportion of patients will nevertheless experience post-surgical recurrence despite treatment with currently available therapies, and these participants carry an even higher risk of recurrence if they undergo further surgery (Fokkens et al 2020, Orlandi R R et al 2013, Agarwal et al 2019, van der Veen et al 2017).

Thymic stromal lymphopoietin (TSLP), an epithelial cell-derived cytokine produced in response to environmental and pro-inflammatory stimuli, leads to the activation of multiple inflammatory cells and downstream pathways (Soumelis et al., 2002; Allakhverdi et al., J Exp Med 2007; 204:253-8). TSLP is increased in the airways of patients with asthma and correlates with Th2 cytokine and chemokine expression (Shikotra et al., J Allergy Clin Immunol 2012; 129:104-11 e1-9) and disease severity (Ying et al., J Immunol 2005; 174:8183-90; Ying, et al. J Immunol 2008; 181:2790-8).

Tezepelumab (also known as AMG 157) (Gilliet, et al., J Exp Med 2003; 197:1059-63) is a fully human monoclonal antibody (immunoglobulin G2A) that targets the thymic, stromal lymphopoietin (TSLP), an epithelial-cell-derived cytokine that promotes inflammatory responses to environmental stimuli through its activities on multiple pathways, including (but not limited to) activities on dendritic cells (Gilliet, et al., 2003; Soumelis et al., 2002; Reche, et al. J Immunol 2001; 167:336-43) and mast cells (Allakhverdi et al., 2007). By binding to TSLP, tezepelumab prevents its interaction with the TSLP receptor complex and inhibits multiple downstream inflammatory pathways.

### SUMMARY

The anti-TSLP antibody described herein addresses an unmet need in patients with chronic rhinosinusitis, with or without nasal polyps, in which other medications may not control symptoms. For example, the antibody therapy may improve nasal polyp score, loss of smell, or reduce the need for surgery to remove polyps in patients with chronic rhinosinusitis, as well as reduce the need for alternate therapies such as systemic corticosteroids.

The disclosure provides, in part, a method for treating chronic rhinosinusitis in a subject comprising selecting a subject in need of treatment for chronic rhinosinusitis, administering a therapeutically effective amount of an anti-TSLP antibody or antibody variant to the subject, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO: 2; or wherein both binding sites of the antibody have identical binding to TSLP, and the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO: 11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO: 9; iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b), wherein the antibody specifically binds to a TSLP polypeptide as set forth in amino acids 29-159 of SEQ ID NO:2.

Also provided is a method for treating chronic rhinosinusitis in a subject comprising administering a composition comprising an anti-TSLP antibody in a dose of 140 mg to 420 mg at an interval of every 2 weeks or every 4 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2; or wherein both binding sites of the antibody have identical binding to TSLP, and the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO: 11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

The disclosure further provides a method for treating chronic rhinosinusitis in a subject comprising selecting a subject in need of treatment for chronic rhinosinusitis, administering a composition comprising an anti-TSLP antibody to the subject in a dose of 140 mg to 420 mg at an interval of every 2 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

Also provided is a method for treating chronic rhinosinusitis in a subject comprising selecting a subject in need of treatment for chronic rhinosinusitis, administering a composition comprising an anti-TSLP antibody to the subject in a dose of 140 mg to 420 mg at an interval of every two weeks, wherein the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

In various embodiments, the antibody is administered every 4 weeks. In various embodiments, the antibody is administered at a dose of 140 mg or at a dose of 420 mg every 2 weeks or every 4 weeks. In various embodiments, the antibody is administered at a dose of 140 mg or at a dose of 210 mg every 2 weeks or every 4 weeks.

In various embodiments, the antibody is administered at a dose of 210 mg every 2 weeks. In various embodiments, the antibody is administered at a dose of 210 mg every 4 weeks.

In various embodiments, the subject is also receiving treatment with corticosteroids.

In various embodiments, provided is a method for treating chronic rhinosinusitis in a subject comprising selecting a subject in need of treatment for chronic rhinosinusitis, administering a therapeutically effective amount of a systemic corticosteroid and a composition comprising an anti-TSLP antibody to the subject in a dose of 210 mg at an interval of every 2 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO: 8, wherein the antibody specifically binds to a TSLP polypeptide as set forth in amino acids 29-159 of SEQ ID NO:2.

In various embodiments, provided is a method for treating chronic rhinosinusitis in a subject comprising selecting a subject in need of treatment for chronic rhinosinusitis, administering a therapeutically effective amount of a systemic corticosteroid and a composition comprising an anti-TSLP antibody to the subject in a dose of 210 mg at an interval of every 2 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO: 8, wherein the antibody specifically binds to a TSLP polypeptide as set forth in amino acids 29-159 of SEQ ID NO:2.

The disclosure also provides a method for treating chronic rhinosinusitis in a subject comprising selecting a subject in need of treatment for chronic rhinosinusitis, administering a composition comprising an anti-TSLP antibody to the subject in a dose of 210 mg at an interval of every 4 weeks, wherein both the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

The disclosure further provides a method for treating chronic rhinosinusitis in a subject comprising selecting a subject in need of treatment for chronic rhinosinusitis, administering a composition comprising an anti-TSLP antibody to the subject in a dose of 210 mg at an interval of every 4 weeks, wherein the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO: 11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

In various embodiments, the light chain variable domain is set out in SEQ ID NO: 12 and the heavy chain variable domain is set out in SEQ ID NO: 10.

In various embodiments, the anti-TSLP antibody or variant has substantially similar pK characteristics as tezepelumab in humans.

In various embodiments, the antibody or antibody variant is administered for a period of at least 4 months, 6 months, 9 months, 1 year or more.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a Fab fragment, an IgG1 antibody, an IgG2 antibody, an IgG3 antibody, and an IgG4 antibody.

In various embodiments, the anti-TSLP antibody thereof is bivalent and selected from the group consisting of a human antibody, a humanized antibody, a chimeric antibody, a monoclonal antibody, a recombinant antibody, an IgG1 antibody, an IgG2 antibody, an IgG3 antibody, and an IgG4 antibody.

In one embodiment, the anti-TSLP antibody variant is selected from the group consisting of a Fab fragment, a single domain antibody, an scFv, wherein the dose is adjusted such that the binding sites are equimolar to those dosed by bivalent antibodies.

In various embodiments, the antibody is an IgG2 antibody.

In one embodiment, the antibody or antibody variant is a human or humanized antibody.

In various embodiments, the antibody is tezepelumab. In various embodiments, the anti-TSLP antibody is an IgG2 antibody having the full length heavy and light chain amino acid sequences set out in SEQ ID NOs: 13 and 14, respectively. In various embodiments, the anti-TSLP antibody is tezepelumab.

In various embodiments, the antibody or antibody variant further is in a pharmaceutical composition further comprising a pharmaceutically acceptable carrier or excipient.

In various embodiments, the antibody is an anti-TSLP antibody described in the art. Exemplary antibodies are described further in the Detailed Description.

In various embodiments, the subject is an adult. In various embodiments, the subject is a child or adolescent.

It is further provided that administration of the anti-TSLP antibody or antibody variant decreases levels of Th2 cytokines in the subject.

In various embodiments, administration of the anti-TSLP antibody or antibody variant improves one or more of the following measures of chronic rhinosinusitis: Nasal Polyp Score (NPS), Nasal Congestion Score (NCS), loss of smell, SinoNasal Outcome Test 22 Item (SNOT-22), EuroQOL quality of life 5-dimensions 3-level version (EQ-5D-3L), Asthma Control Questionnaire (ACQ-6), Lund-Mackay score, modified Lund-Mackay Score, incidence of nasal polyp surgery and/or systemic corticosteroids (SCS).

In various embodiments, the NPS and or the NCS is reduced by one, two or three points. In various embodiments, the subject has an NPS score of 0 (none) or 1 (mild) at week 52 after treatment. In various embodiments, the subject has a starting NPS score of 2. In various embodiments, the subject has a starting NPS score of 3. In various embodiments, the subject has an NCS score of 0 (none) or 1 (mild) at week 52 after treatment. In various embodiments, the subject has a starting NCS score of 2. In various embodiments, the subject has a starting NCS score of 3.

In various embodiments, treatment with anti-TSLP modulates the levels of one or more of the following biomarkers of chronic rhinosinusitis: cytokines, IgE, CCL17, CCL18, CCL22, and RNA transcriptional changes in nasal epithelium. In various embodiments, treatment with anti-TSLP reduces the level of Th2 cytokines. In various embodiments, the treatment modulates (reduces or moderates) levels of or activity of one or more of IL-4, IL-5, IL-13, IL-17, IL-22, IL-23, IL-31, or combinations thereof.

Provided herein are methods for treating chronic rhinosinusitis with nasal polyps in a subject comprising selecting a subject in need of treatment for chronic rhinosinusitis with nasal polyps, administering a composition comprising an anti-TSLP antibody to the subject in a dose of 210 mg at an interval of every 4 weeks, wherein the antibody comprises
A. a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8; or,
B. a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO: 11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO: 2.

Further provided is a method for treating chronic rhinosinusitis in a subject comprising selecting a subject in need of treatment for chronic rhinosinusitis, administering a composition comprising an anti-TSLP antibody to the subject in a dose of 140 to 420 mg at an interval of every 2 or every 4 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2, wherein the antibody is an IgG2 antibody.

In various embodiments, the light chain variable domain is set out in SEQ ID NO: 12 and the heavy chain variable domain is set out in SEQ ID NO: 10.

In various embodiments, the antibody is administered every 4 weeks. In various embodiments, the antibody is administered at a dose of 210 mg every 2 weeks or every 4 weeks.

In various embodiments, the antibody is administered at a dose of 210 mg every 4 weeks.

Also provided is a method of reducing the frequency of surgery needed or systemic corticosteroids in a subject having chronic rhinosinusitis, with or without nasal polyps, comprising selecting a subject in need of treatment for chronic rhinosinusitis, administering a composition comprising of an anti-TSLP antibody to the subject in a dose of 140 mg to 420 mg at an interval of every 2 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

Also provided is a method of reducing the frequency of surgery needed or systemic corticosteroids in a subject having chronic rhinosinusitis, with nasal polyps, comprising selecting a subject in need of treatment for chronic rhinosinusitis, administering a composition comprising of an anti-TSLP antibody to the subject in a dose of 140 mg to 420 mg at an interval of every 2 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

Further provided is a method of reducing the frequency of chronic rhinosinusitis exacerbation in a subject comprising selecting a subject in need of treatment for chronic rhinosinusitis, administering a composition comprising an anti-TSLP antibody to the subject in a dose of 140 mg to 420 mg at an interval of every 2 weeks, wherein the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

Also provided is a method of reducing the frequency of chronic rhinosinusitis exacerbation in a subject comprising selecting a subject in need of treatment for chronic rhinosinusitis, administering a composition comprising an anti-TSLP antibody to the subject in a dose of 210 mg at an interval of every 4 weeks, wherein the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

Also provided are methods for reducing SNOT-22 and/or Lund-Mackay score in a subject comprising administering a composition comprising an anti-TSLP antibody in a dose of 140 mg to 420 mg at an interval of every 2 weeks, or at a dose of 210 mg at an interval or every 4 weeks, wherein the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

In various embodiments, the disclosure provides a method for reducing SNOT-22 and/or Lund-Mackay score in a subject comprising administering a composition comprising an anti-TSLP antibody in a dose of 140 mg to 420 mg at an interval of every 2 weeks, or at a dose of 210 mg at an interval or every 4 weeks, the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

In various embodiments, the disclosure provides a method for reducing a SNOT-22 domain score in a subject comprising administering a composition comprising an anti-TSLP antibody in a dose of 140 mg to 420 mg at an interval of every 2 weeks, or at a dose of 210 mg at an interval or every 4 weeks, wherein the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

In various embodiments, the SNOT-22 domain is Nasal Domain, Ear/Facial Domain, Sleep Domain, Function Domain or Emotion Domain. In various embodiments, the administration ameliorates one or more symptoms of the domain score selected from: need to blow nose, nasal blockage, sneezing, runny nose, cough, post-nasal discharge, thick nasal discharge, decreased sense of smell/taste; ear fullness, dizziness, ear pain, facial pain/pressure; difficulty falling asleep, wake up at night, lack of a good night's sleep, wake up tired; fatigue, reduced productivity, reduced concentration, frustrated/restless/irritable, sadness, and embarrassment.

In various embodiments, the antibody comprises a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO: 11; and b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

In various embodiments, the light chain variable domain is set out in SEQ ID NO: 12 and the heavy chain variable domain is set out in SEQ ID NO: 10.

In various embodiments, the antibody is tezepelumab.

It is provided that the dosing and antibody and antibody variant types referenced above apply to each method contemplated herein.

In various embodiments, provided is a pharmaceutical composition comprising an antibody or antibody variant and further comprising a pharmaceutically acceptable carrier or excipient.

In various embodiments, the administration delays the time to a chronic rhinosinusitis exacerbation compared to a subject not receiving the anti-TSLP antibody or a patient's baseline symptoms prior to receiving treatment with an anti-TSLP antibody or antibody variant. Examples of such an antibody include tezepelumab.

In various embodiments, the administration delays the incidence or time to nasal polyp surgery in a subject with chronic rhinosinusitis compared to a subject not receiving the anti-TSLP antibody.

Administration of just TSLP antibody or antibody variant can be done subcutaneously.

In various embodiments, the administration reduces frequency of or levels of co-administered therapy in the subject. Exemplary co-administered therapy is dupilumab, immunosuppressive or immunomodulating drugs (e.g., systemic corticosteroids, cyclosporine, mycophenolate-mofetil, interferon (IFN)-gamma, Janus kinase inhibitors, azathioprine, methotrexate), anti-IL-13 antibodies, anti-IL-5 pathway antibodies (benralizumab, mepolizumab, reslizumab), or combinations thereof. “Co-administration” for this purpose would cover the administration of two different molecules sited above in the same syringe/autoinjector, or two different syringe/autoinjectors. The different molecules above can also be administered at the same time or at different times.

In various embodiments, the administration reduces or eliminates the need for corticosteroid therapy.

In various embodiments, the administration is subcutaneous or intravenous.

In various embodiments, the antibody is tezepelumab or another anti-TSLP antibody or antibody variant described in the art, e.g., in Table A or in WO 20220226342A1, WO 20220226339A1, WO2023098491A1, WO2021155634A1, WO2022166072A1, WO2021043221A1, WO2022184074A1, WO2021104053A1, WO2023116925A1, WO2021155861A1, WO2022116858A1, WO2022117079A1, WO2020244544A1, WO2021152488A1, WO2022253147A1, WO2023070948A1, WO2023142309A1, WO2022095689A1, WO2021115240A1, WO2022166739A1 and WO2019100111A1. Exemplary antibodies are described further in the Detailed Description.

### BRIEF DESCRIPTION OF THE DRAWINGS

FIG. 1A and FIG. 1B. Baseline demographics and clinical characteristics of subjects with chronic rhinosinusitis, with or without nasal polyps, treated with anti-TSLP antibody.

FIG. 2. LS mean change from baseline to week 52 of SNOT-22 score in patients with a history of nasal polyps treated with anti-TSLP antibody.

FIG. 3. Mean change from baseline to week 52 of SNOT-22 score in patients with any history of nasal polyps treated with anti-TSLP antibody.

FIG. 4. Improvements from baseline to week 52 in SNOT-22 domain scores with tezepelumab compared with placebo in patients with a history of NP. Placebo, n=57; tezepelumab 210 mg Q4W, n=65. n is the number of patients contributing to the analysis, i.e., the number of patients with a baseline value and non-missing independent variables included in the model. CI, confidence interval; LS, least-squares; NP, nasal polyp; SNOT-22, Sino-Nasal Outcome Test-22.

FIGS. 5A-5G. Changes from baseline in SNOT-22 total and domain scores in patients with a history of NP. FIG. 5A, Total Score %; FIG. 5B, total score; FIG. 5C, Ear/Facial Domain; FIG. 5D, Emotion Domain; FIG. 5E, Function Domain; FIG. 5F, Nasal Domain; FIG. 5G, Sleep Domain. Baseline: placebo, n=62; tezepelumab, n=69. Week 28: placebo, n=57; tezepelumab, n=64. Week 52: placebo, n=53; tezepelumab, n=64. LS, least-squares; NP, nasal polyp; Q4W, every 4 weeks; SE, standard error; SNOT-22, Sino-Nasal Outcome Test-22.

FIGS. 6A-6E. Changes from baseline to week 52 in SNOT-22 item scores of interest in patients with a history of NP. FIG. 6A, Decreased sense of smell/taste; FIG. 6B, Nasal blockage; FIG. 6C, Cough; FIG. 6D, Reduced productivity; FIG. 6E, Waking up tired. n is the number of patients contributing to the analysis, i.e., the number of patients with a baseline value and non-missing independent variables included in the model. CI, confidence interval; LS, least-squares; NP, nasal polyp; Q4W, every 4 weeks; SE, standard error; SNOT-22, Sino-Nasal Outcome Test-22.

FIG. 7. Sequences of TSLP and anti-TSLP antibody tezepelumab.

### DETAILED DESCRIPTION

Use of an anti-TSLP antibody addresses an unmet need in chronic rhinosinusitis (CRS) or chronic rhinosinusitis with nasal polyps (CRSwNP) patients in which other medications may not control moderate to severe symptoms. It is further provided that treatment with anti-TSLP antibodies such as tezepelumab could eliminate regular disease activity and make more patients steroid-free or reduce the need for use of steroids in the treatment of CRS and CRSwNP.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “chronic rhinosinusitis” as used herein refers to inflammatory condition of the nasal and paranasal mucosa which is believed that type 2 (T2) driven inflammation is a dominant process in CRS. Chronic rhinosinusitis can be chronic rhinosinusitis with nasal polyps (CRSwNP) or without nasal polyps (CRSsNP) and may also be observed with or without co-morbid asthma/allergy, asthma, infection and aspirin sensitivity-exacerbated respiratory disease (AERD) or Nonsteroidal anti-inflammatory drug exacerbated respiratory disease (NSAID-ERD).

The term “chronic rhinosinusitis exacerbation” as used herein refers to a worsening of chronic rhinosinusitis that leads to any of the following: Use of systemic corticosteroids or other adjunct therapy for at least 3 days; worsening of one or more symptoms or measures of chronic rhinosinusitis described herein, and/or growth or expansion of nasal polyp number, size or severity.

The term “worsening of chronic rhinosinusitis” refers to new or increased symptoms and/or signs (examination) that can be either concerning to the subject having chronic rhinosinusitis with or without nasal polyps (subject-driven) or related to a Patient Daily Diary alert (diary-driven).

The term “cytokine” as used herein refers to one or more small (5-20 kD) proteins released by cells that have a specific effect on interactions and communications between cells or on the behavior of cells, such as immune cell proliferation and differentiation. Functions of cytokines in the immune system include, promoting influx of circulating leukocytes and lymphocytes into the site of immunological encounter; stimulating the development and proliferation of B cells, T cells, peripheral blood mononuclear cells (PBMCs) and other immune cells; and providing antimicrobial activity. Exemplary immune cytokines, include but are not limited to, IL-1, IL-2, IL-3, IL-4, IL-5, IL-6, IL-7, IL-9, IL-10, IL-12, IL-13, IL-15, IL17A, IL-17F, IL-18, IL-21, IL-22, IL-23, IL-31, IL-33, interferon (including IFN alpha, beta, and gamma), tumor necrosis factor (including TNF alpha, beta), transforming growth factor (including TGF alpha, beta), granulocyte colony stimulating factor (GCSF), granulocyte macrophage colony stimulating factor (GMCSF) and thymic stromal lymphopoietin (TSLP).

A “T helper (Th) 1 cytokine” or “Th1-specific cytokine” refers to cytokines that are expressed (intracellularly and/or secreted) by Th1 T cells, and include IFN-g, TNF-α, and IL-12. A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-a, IL-2 and IL-17.

The term “specifically binds” is “antigen specific”, is “specific for”, “selective binding agent”, “specific binding agent”, “antigen target” or is “immunoreactive” with an antigen refers to an antibody or polypeptide that binds a target antigen with greater affinity than other antigens of similar sequence. It is provided herein that the agent specifically binds target proteins useful in identifying immune cell types, for example, a surface antigen (e.g., T cell receptor, CD3), a cytokine (e.g., TSLP, IL-4, IL-5, IL-13, IL-17, IFN-g, TNF-α) and the like. In various embodiments, the antibody specifically binds the target antigen, but can cross-react with an ortholog of a closely related species, e.g., an antibody may be a human protein and also bind a closely related primate protein.

The term “antibody” or “immunoglobulin” refers to a tetrameric glycoprotein that consists of two heavy chains and two light chains, each comprising a variable region and a constant region. “Heavy Chains” and “Light Chains” refer to substantially full-length canonical immunoglobulin light and heavy chains (see e.g., Immunobiology, 5th Edition (Janeway and Travers et al., Eds., 2001). Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies.

Antibody variants include antibody fragments and antibody like proteins with changes to structure of canonical tetrameric antibodies. Typically antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody fragments include antigen-binding portions of the antibody including, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Valency” refers to the number of antigen binding sites on each antibody or antibody fragment that targets an epitope. A typical full length IgG molecule, or F(ab)₂ is “bivalent” in that it has two identical target binding sites. A “monovalent” antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity” includes inhibiting any one or more of the following: binding of TSLP to its receptor; proliferation, activation, or differentiation of cells expressing TSLPR in the presence of TSLP; inhibition of Th2 cytokine production in a polarization assay in the presence of TSLP; dendritic cell activation or maturation in the presence of TSLP; and mast cell cytokine release in the presence of TSLP. See, e.g., U.S. Pat. No. 7,982,016 B2, column 6 and example 8 and US 2012/0020988 A1, examples 7-10.

“Tezepelumab” refers to an antibody that has the same amino acid sequence of the INN tezepelumab-ekko. Tezepelumab also has the heavy and light chain amino acid sequences set out in SEQ ID NO: 13 and 14, respectively.

The term “sample” or “biological sample” refers to a specimen obtained from a subject for use in the present methods, and includes urine, whole blood, plasma, serum, saliva, sputum, skin or tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

Chronic Rhinosinusitis with Nasal Polyps (CRSwNP)

Chronic rhinosinusitis with nasal polyps (CRSwNP) is a chronic heterogeneous inflammatory condition of the nasal and paranasal mucosa (Fokkens et al 2020). The underlying pathogenesis of the disease is currently undefined but in recent years type 2 (T2) driven inflammation has been shown to be a dominant process in CRSwNP and therapeutics targeting T2 inflammation have demonstrated efficacy in CRSwNP (Gevaert et al 2011, Bachert et al 2016, Bachert et al 2017, Bachert et al 2019, Gevaert et al 2020). Thymic stromal lymphopoietin (TSLP) is an epithelial cytokine released in response to airborne environmental triggers. TSLP can drive T2 cytokine release from Th2 and ILC2 cells as well as have effects on other immune cells present in NP tissue. TSLP levels are elevated in nasal polyp tissue from patients with CRSwNP compared with healthy sinus tissue or that from patients with CRS without NP (Kimura S et al 2011, Nagarkar D R et al 2013). TSLP may therefore play a role in the initiation and persistence of CRSwNP and blockade is predicted to be beneficial to patients.

Currently available biological treatment for NP is limited to an approximately 40% to 60% success rate (Gevaert et al 2011, Bachert et al 2016, Bachert et al 2017, Bachert et al 2019, Gevaert et al 2020). There are two possible explanations for the lack of complete response with biologics. First is that when polyps become large enough to be symptomatic, reduction of inflammatory cells and/or inflammation may limit and reduce new growth but may not be able to substantially reduce polyp size in the face of persistent tissue fibrosis (Stevens et al 2016). TSLP contribution to extracellular matrix (ECM) deposition may be indirect, as an initiator of the inflammatory processes driving fibrosis, or direct, since TSLP has been shown to have effects on or to be produced by lung fibroblasts (reviewed in Gauvreau et al 2020). Furthermore, in CRSwNP TSLP has been shown to indirectly stimulate fibroblasts via CST-1 (Kato et al 2019) inducing periostin; a matricellular signalling protein demonstrated to regulate ECM structure and organization through its ability to bind ECM and promote collagen fibrillogenesis and crosslinking. This suggests that in addition to its anti-inflammatory MOA treatment with tezepelumab may potentially have an anti-fibrotic effect which could result in improvement of CRSwNP. The kinetics of ECM turnover is known to be slow therefore the 52-week treatment period proposed is required if sufficient time is to be given for changes in ECM to occur. Continued improvement in NPS score beyond week 24 may be expected based on this potential anti-fibrotic MOA for tezepelumab.

Second, there is a subset of CRSwNP patients who do not have T2 mediated disease. Analysis of subgroups in the tezepelumab Phase 2b study and Phase 3 studies (PATHWAY, NCT02054130 and NAVIGATOR, NCT03347279) in asthma patients showed that tezepelumab benefited participants with low and high baseline eosinophil counts and a low and high baseline T2 profile. These analyses suggest that tezepelumab, unlike other currently available biologics on the market used to treat severe asthma, is effective in patients with T2 high and T2 low airway inflammation and might be effective in T2 low as well as T2 high CRSwNP. It is hypothesized that tezepelumab reduces the AAER in asthma by acting as an airway anti-inflammatory.

The eosinophilic (Type 2 high) endotype of CRSwNP is most common in the United States, Europe, and Japan while non-eosinophilic endotype is more frequent in other Asian countries (Lou H et al 2018, Hull and Chandra 2017, Fokkens et al 2020, Shin S H et al 2014, Chitsuthipakorn W et al 2018).

CRSwNP is strongly linked with asthma by epidemiological, pathophysiological, and clinical evidence, supporting an “united airway” concept to chronic inflammatory diseases of the upper and lower airways. Both diseases are comorbid conditions (Håkansson K et al 2015). Compared to non-asthmatic patients, asthmatic patients have a higher prevalence of CRS and CRSwNP and greater severity of rhinosinusitis (Jarvis et al 2012, Seybt et al 2007, Pearlman et al, 2009, Tint et al 2016, Settipane 1977). Furthermore, asthmatic subjects with CRSwNP may have more poorly controlled asthma, increased airway obstruction, and greater lower airway inflammation than those without CRSwNP (Bilodeau et al 2010). Co-existing asthma in CRSwNP patients is associated with lower quality of life (Alobid I et al 2005, Ehnhage A et al 2009). Mechanistically, both NP and severe eosinophilic asthma are considered Type-2 (T2) driven diseases and the inflammatory profile is similar between the upper airway in NP and the lower airway in asthma (Fokkens et al 2020, Chaaban M R et al 2013). Like asthma, the role of eosinophils is considered important to the pathology of NP in a high proportion of patients.

Asthma and CRSwNP share some pathophysiology and common comorbidities. A post-hoc analysis of patients with comorbid nasal polyps in the PATHWAY study population revealed that 15.2% of the subjects had comorbid CRSwNP. Asthma patients with NP treated with tezepelumab demonstrated improvement in AERR, FEV1 and ACQ-6 and T2 inflammatory biomarkers relative to placebo to an equivalent extent as non-NP asthma patients. These finding support the rationale of a broad effect of tezepelumab in asthma and potential efficacy in CRSwNP. (Emson C et al, 2020; Emson C et al, 2021).

The etiology of NP is still considered unclear, but allergy, asthma, infection and aspirin sensitivity (AERD) have all been associated with this complex refractory disease in adults (Hull and Chandra 2017). Both NP and asthma are T2-driven disease processes that are characterized by an eosinophilic infiltrate in affected tissue, either the polyps or the airways. The inflammatory profile observed in asthma shares many features and similarities with the inflammation seen in patients with NP where eosinophils are the primary effector cell in the pathophysiology of both upper airway sinusitis and lower airway asthma (Håkansson K et al 2015; Ediger et al 2005).

Nasal polyposis is recognized to be a consequence of chronic inflammatory disease of the nasal mucosa. The presence of polyps can cause long-term symptoms such as prominent nasal obstruction, post-nasal drip and nasal discharge, loss of smell, and facial pain. These symptoms can greatly impact a patient's quality of life. Patients with CRSwNP have been observed to have worse symptoms (as evidenced by higher Sino-Nasal Outcome Test [SNOT]-22 scores), more severe disease by imaging (as demonstrated by Lund-Mackay and modified LMK score computed tomography [CT] scores), and require more frequent revision surgery than patients with CRSsNP (Hull and Chandra 2017; Fokkens et al 2020).

Nasal polyposis represents an area of significant unmet medical need, especially for a subset of patients with NP who have exhausted current treatment options, which include medical interventions (intranasal corticosteroids (INCS), systemic corticosteroids (SCS), and antibiotics) and surgical interventions. Patients and healthcare professionals both point to a greater need for new therapies that reduce the need for repeated courses of SCS or surgery for all patients with NP. Surgery to remove polyps remains a consideration in patients whose symptoms are not controlled with corticosteroids, but rates of recurrence are high (approximately 40%, 12 months after surgery) and repeated surgical interventions are often needed. Although INCS are continued after surgery, a proportion of patients will nevertheless experience post-surgical recurrence despite treatment with currently available therapies, and these participants carry an even higher risk of recurrence if they undergo further surgery (Fokkens et al 2020, Orlandi R R et al 2013, Agarwal et al 2019, van der Veen et al 2017).

Chronic rhinosinusitis without nasal polyps (CRSsNP) is also detected in patients with allergic and non-allergic upper and lower airway diseases, epithelial cell disorders, immunodeficiencies, autoimmune diseases, certain infectious diseases, those with higher incidence of abnormal biofilms, and innate immune defects (Cho et al., J Allergy Clin Immunol Pract. 2016 4 (4): 575-582). Characterization of subjects having CRSsNP is described in “Chronic Rhinosinusitis Without Nasal Polyps: Clinical Characteristics And Comorbid Diseases”, The Journal Of Allergy And Clinical Immunology: In Practice 2018.

Chronic rhinosinusitis can be assessed using several objective and subjective measurements such as the Nasal Polyp Score and Nasal Congestion Score. The measurements allow investigators to assess overall disease severity at one given time point and consists of a 4-point severity scale from clear to severe disease (0=non disease; 1=mild; 2=moderate disease; 3=severe disease). The NPS is the sum of the right and left nostril scores (maximum 8), as evaluated by nasal endoscopy. Total NPS is graded based on polyp size described in Table 2.

Additional measures of chronic rhinosinusitis severity/improvement include Nasal Polyposis Symptom Diary (NPSD) and Nasal Polyposis Symptom Screening Assessment (NPSSA), loss of smell, SinNasal Outcome Test 22 Item (SNOT-22), EuroQOL quality of life 5-dimensions 3-level version (EQ-5D-3L), Asthma Control Questionnaire (ACQ-6), Lund-Mackay score, modified Lund-Mackay Score, incidence of nasal polyp surgery and/or systemic corticosteroids (SCS).

Nasal Polyposis Symptom Diary (NPSD) and Nasal Polyposis Symptom Screening Assessment (NPSSA) is an 11-item NP symptom diary to be completed each morning throughout the screening, treatment, and follow-up periods. The participant is asked to consider their experience with NP/nasal polyps over the past 24 hours when responding to each question. Participants are asked to report their experience with NP symptoms (nasal blockage, nasal congestion, runny nose, postnasal drip (mucus drainage down the throat), headache, facial pain, facial pressure, and difficulty with sense of smell) and symptom impacts (difficulty with sleeping due to nasal symptoms and difficulty with daily activities due to nasal symptoms). Participants report the severity of each symptom and symptom impact at its worst using a 4-point verbal rating scale (0-None to 3-Severe). A total symptom score (TSS) is calculated by taking the sum of the 8 equally weighted symptom items. A single item to capture INCS compliance (yes or no) will be administered after the symptom and symptom impact item.

Nasal Congestion score (NCS) is captured by an item in the NPSD asking participants to rate the severity of their worst nasal congestion over the past 24 hours using the following response options: 0-None; 1-Mild; 2-Moderate; 3-Severe.

Loss of smell is captured by an item in the NPSD asking participants to rate the severity of their worst difficulty with sense of smell over the past 24 hours using the following response options: 0—None; 1—Mild; 2—Moderate; 3—Severe.

The SNOT-22 is a condition-specific Health-Related Quality of Life (HRQOL) assessment which captures participant-reported physical problems, functional limitations, and emotional consequences of SinoNasal conditions (Piccirillo et al 2002; Hopkins et al 2009). Patient-reported symptom severity and symptom impact are captured via a 6-point scale (0-No Problem to 5-Problem as bad as it can be). The total score is the sum of item scores and has a range from 0 to 110 (higher scores indicate poorer outcomes). A Minimal Clinical Importance Difference (MCID) of 8.90 has been established for individual score change (Hopkins et al 2009) as a clinically meaningful improvement.

Asthma Control Questionnaire (for asthma/AERD/NSAID-ERD participants only): The Asthma Control Questionnaire (ACQ-6) is an assessment of asthma symptoms (night-time waking, symptoms on waking, activity limitation, shortness of breath, wheezing, and short acting beta-agonist use). Participants are asked to recall their level of asthma control during the previous week by responding to one bronchodilator use question and 5 symptom questions. Questions are weighted equally and scored from 0 (totally controlled) to 6 (severely uncontrolled). The mean ACQ-6 score is the mean of the responses. Mean scores of ≤0.75 indicate well-controlled asthma, scores between 0.75 and <1.5 indicate partly controlled asthma, and a score ≥1.5 indicates not well controlled asthma (Juniper et al 2006). Individual changes of at least 0.5 are considered to be a clinically meaningful improvement.

The University of Pennsylvania Smell Identification Test (UPSIT) is a quantitative test of olfactory function which uses microencapsulated odorants that are released by scratching standardized odor-impregnated test booklets (Doty et al 1984). Four booklets each with 10 odorants each are used for the test. Participants are asked to identify the odor using multiple choice format which lists different possibilities. The test is forced-choice, i.e., the participant is required to mark one of the four alternatives even if no smell is perceived. Scores are based on number of correctly identified odors (score range 0 to 40).

The Patient Global Impression of Severity (PGI-S) is a single item designed to capture the participant's perception of overall NP symptom severity at the time of completion using a 6-point categorical response scale (0—No Symptoms to 5—Very Severe). The Patient Global Impression of Change (PGI-C) instrument captures the participant's overall evaluation of response to treatment since first dose of IP. The participant is asked to report the degree to which their health status has changed using a 7-point scale (1-Much Better to 7-Much Worse).

The Work Productivity and Activity Impairment questionnaire (WPAI, General Health version 2.0) is a self-administered tool comprised of 6 questions which address absenteeism, presenteeism (reduced effectiveness while working), overall work productivity loss (absenteeism plus presenteeism), and activity impairment. This validated tool captures data from the past 7 days. WPAI outcomes are scored as impairment percentages, with a higher percentage indicating greater impairment and less productivity (Reilly et al 1993).

The Lund-Mackay score scoring system is used to provide a semi-quantitative assessment of nasal sinuses on sinus CT scans (Lund and Mackay 1993). Based on the sinus CT images, the five sinuses (maxillary, anterior ethmoid, posterior ethmoid, sphenoid and frontal) on each side are scored.

The EuroQOL quality of life 5-dimensions 3-level version (EQ-5D-3L) is a standardized instrument for use as a measure of health-related quality of life (HRQOL) and was developed by EuroQol (Brooks, 1996). It defines health in terms of 5 dimensions: mobility, self-care, usual activities, pain/discomfort, and anxiety/depression. Each dimension has 3 ordinal levels of severity: 1, no problem; 2, some problems; and 3, severe problems. Overall health state is defined as a 5-digit number.

### TSLP

Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+ progenitor cells.⁹ TSLP signals through a heterodimeric receptor consisting of the interleukin (IL)-7 receptor alpha (IL-7Rα) chain and a common γ chain-like receptor (TSLPR) (Pandey, Nat Immunol. 1:59-64, 2000; Park, J Exp Med. 192:659-669, 2000).

Data from other studies suggest that TSLP may promote airway inflammation through Th2 independent pathways such as the crosstalk between airway smooth muscle and mast cells (Allakhverdi et al, J Allergy Clin Immunol. 123 (4): 958-60, 2009; Shikotra et al, supra). TSLP can also promote induction of T cells to differentiate into Th-17-cytokine producing cells with a resultant increase in neutrophilic inflammation commonly seen in more severe asthma (Tanaka et al, Clin Exp Allergy. 39 (1): 89-100, 2009). These data and other emerging evidence suggest that blocking TSLP may serve to suppress multiple biologic pathways including but not limited to those involving Th2 cytokines (IL-4/13/5).

### Antibodies

It is provided that antibodies or antibody variants specific for TSLP are useful in the treatment of chronic rhinosinusitis, including moderate or severe chronic rhinosinusitis, chronic rhinosinusitis with or without nasal polyps, and/or chronic rhinosinusitis with or without Asthma/AERD/NSAID-ERD.

It is provided that antibodies or antibody variants specific for TSLP are useful in the treatment of chronic rhinosinusitis, including moderate or severe chronic rhinosinusitis, chronic rhinosinusitis with nasal polyps, and/or chronic rhinosinusitis with or without Asthma/AERD/NSAID-ERD.

Specific binding agents such as antibodies and antibody variants or fragments that bind to their target antigen, e.g., TSLP, are useful in the methods of the disclosure. In one embodiment, the specific binding agent is an antibody. The antibodies may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody. For example, with CDR-grafting, non-human CDR sequences are inserted into human framework regions (Lu et al. Journal of Biomedical Science (2020) 27:1).

Also encompassed by the disclosure are human antibodies and antibody variants (including antibody fragments) that bind TSLP. Human antibody refers to an antibody generated from human immunoglobulin sequences and comprising human variable and constant regions. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Alternatively, a “human” antibody can be made by using human constant regions and framework regions and synthetically making the human antibody. Human antibodies can also be produced by the expression of recombinant DNA in host cells or by expression in hybridoma cells as described herein, using phage display libraries, or by single B cell cloning (Lu et al. Journal of Biomedical Science (2020) 27:1). It is provided that human, humanized, or chimeric antibodies may comprise mutations or modification that enhance stability of the antibody or extend antibody half-life, e.g., SEFL, YTE, and other modifications known in the field (See e.g., Wang et al., Protein Cell. 2018 9:63-73).

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein. Additional mutations can be made to human, humanized or chimeric antibodies to improve stability or half-life. Such mutations are known in the art.

Antibodies and antibody variants (including antibody fragments) useful in the present methods comprise an anti-TSLP antibody comprising a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and, b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody or antibody variant specifically binds to a TSLP polypeptide as set forth in amino acids 29-159 of SEQ ID NO:2.

Also provided is an antibody or antibody variant comprising a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO: 11; iii. a sequence of amino acids encoded by a polynucleotide that hybridizes under moderately stringent conditions to the complement of a polynucleotide consisting of SEQ ID NO: 11; and, b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody or antibody variant specifically binds to a TSLP polypeptide as set forth in amino acids 29-159 of SEQ ID NO: 2.

Tezepelumab is an exemplary anti-TSLP antibody having: a. i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8.

Tezepelumab also comprises a light chain variable domain having the amino acid sequence set out in SEQ ID NO: 12; encoded by a polynucleotide sequence set out in SEQ ID NO: 11; and a heavy chain variable domain having the amino acid sequence set out in SEQ ID NO: 10, encoded by a polynucleotide sequence set out in SEQ ID NO:9.

Tezepelumab is an IgG2 antibody. The sequence of the full-length heavy chain and light chain of tezepelumab, including the IgG2 chain, is set out in SEQ ID NOs: 13 and 14, respectively.

Exemplary sequences of tezepelumab are also set out in U.S. Pat. No. 7,982,016 SEQ ID NOs: 13, 60, 105, 145, 173, 212; SEQ ID NOs: 361 and 363; and a light chain comprising a light chain variable domain comprising the amino acid sequence as set forth in SEQ ID NO:363 and a lambda light chain constant domain comprising the amino acid sequence as set forth in SEQ ID NO:369; and a heavy chain comprising a heavy chain variable domain comprising the amino acid sequence as set forth in SEQ ID NO:361 and an IgG2 heavy constant domain comprising the amino acid sequence as set forth in SEQ ID NO: 365, herein incorporated by reference.

In various embodiments, anti-TSLP antibody derivatives are provided for use in the methods. Anti-TSLP antibody derivatives are described in International Patent Publications WO 2022/226342 and WO 2022/226339, incorporated herein by reference in their entireties. Exemplary derivatives include tezepelumab antibodies that may have modifications after purification of antibody or after long term storage, including but not limited to, an isomerization derivative, a deamidation derivative, an oxidation derivative, a glycosylation derivative, disulfide isoform derivatives, and/or high molecular weight (HMW) species or antibody fragments. Exemplary derivatives may also be variants that have the amino acid sequences set out in SEQ ID NOs: 13-36 of WO2022/226342 as well as variants therein of SEQ ID NO: 3-8, e.g., including residues identified as possible sources of reduced stability in anti-TSLP antibody tezepelumab CDRs (SEQ ID NOs: 3-8) or in the variable region (SEQ ID NOs: 10 and 12), which include CDRH1 M34, CDRH2 W52, CDRH2 D54, CDRH2 N57, CDRH2 D62, CDRH3 W102, FRH1 N25, FRH1 N26, CDRL2 D49, CDRL2 D50, FRL2 N65, CDRL3 W90, CDRL3 D91, CDRL3 S92, S93, S94, and/or CDRL3 D95.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a Fab fragment, an IgG1 antibody, an IgG2 antibody, an IgG3 antibody, and an IgG4 antibody.

In various embodiments, the anti-TSLP antibody is bivalent and selected from the group consisting of a human antibody, a humanized antibody, a chimeric antibody, a monoclonal antibody, a recombinant antibody, an IgG1 antibody, an IgG2 antibody, an IgG3 antibody, and an IgG4 antibody.

In various embodiments, the anti-TSLP antibody variant is selected from the group consisting of a Fab fragment, single domain antibody, scFv, wherein the dose is adjusted such that the binding sites to be equimolar to the those dosed by bivalent antibodies.

It is provided that the antibody is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, to increase or decrease the affinity of the antibodies to human TSLP described herein, or to increase stability or extend half-life (such as YTE mutations).

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

### Methods of Administration

In one aspect, methods of the present disclosure include a step of administering a therapeutic anti-TSLP antibody or antibody variant described herein, optionally in a pharmaceutically acceptable carrier or excipient. In certain embodiments, the pharmaceutical composition is a sterile composition.

Provided herein are methods method for treating chronic rhinosinusitis or chronic rhinosinusitis in a subject, including severe or moderate chronic rhinosinusitis, chronic rhinosinusitis with or without nasal polyps, and/or chronic rhinosinusitis with asthma, AERD or NSAID-ERD. In various embodiments, the method comprises selecting a subject in need of treatment for chronic rhinosinusitis, and administering an anti-TSLP antibody as described herein. In various embodiments, the chronic rhinosinusitis is with nasal polyps. In various embodiments, the subject has moderate or severe asthma. In various embodiments, the subject has moderate asthma.

In various embodiments, the antibody is tezepelumab or another anti-TSLP antibody described in the art. Exemplary anti-TSLP antibodies include antibodies described in WO 2017/042701, WO 2016/142426, WO 2010/017468, US20170066823, US20120020988 and U.S. Pat. No. 8,637,019, incorporated herein by reference, some of which are described below in Table A. In exemplary aspects, the anti-TSLP antibody is selected from an antibody of Table A and antibodies or antibody fragments described in WO 20220226342A1, WO 20220226339A1, WO2023098491A1, WO2021155634A1, WO2022166072A1, WO2021043221A1, WO2022184074A1, WO2021104053A1, WO2023116925A1, WO2021155861A1, WO2022116858A1, WO2022117079A1, WO2020244544A1, WO2021152488A1, WO2022253147A1, WO2023070948A1, WO2023142309A1, WO2022095689A1, WO2021115240A1, WO2022166739A1 and WO2019100111A1, the disclosure of each of which is incorporated by reference herein.

TABLE A

| | |
|---|---|
| WO2017/042701 | An anti-TSLP antibody comprising a heavy chain (HC) CDR1 comprising |
| | the sequence of SEQ ID NO: 13, a HC CDR2 comprising the sequence |
| | of SEQ ID NO: 14, and a HC CDR3 comprising the sequence of SEQ ID |
| | NO: 15; |
| | An anti-TSLP antibody comprising a light chain (LC) CDR1 comprising |
| | the sequence of SEQ ID NO: 16, a LC CDR2 comprising the sequence |
| | of SEQ ID NO: 17, a LC CDR3 comprising the sequence of SEQ ID NO: |
| | 18; |
| | An anti-TSLP antibody comprising a heavy chain (HC) CDR1 comprising |
| | the sequence of SEQ ID NO: 19, a HC CDR2 comprising the sequence |
| | of SEQ ID NO: 20, a HC CDR3 comprising the sequence of SEQ ID |
| | NO: 15; |
| | An anti-TSLP antibody comprising a light chain (LC) CDR1 comprising |
| | the sequence of SEQ ID NO: 21, a LC CDR2 comprising the sequence |
| | of SEQ ID NO: 22, a LC CDR3 comprising the sequence of SEQ ID NO: |
| | 23; |
| | An anti-TSLP antibody comprising a HC variable region comprising the |
| | sequence of SEQ ID NO: 26 and/or a LC variable region comprising the |
| | sequence of SEQ ID NO: 27; |
| | An anti-TSLP antibody comprising a HC variable region comprising the |
| | sequence of SEQ ID NO: 28 and/or a LC variable region comprising the |
| | sequence of SEQ ID NO: 29; |
| | An anti-TSLP antibody that comprises a paratope comprising at least |
| | one of the following residues: Thr28, Asp31, Tyr32, Trp33, Asp56, |
| | Glu101, Ile102, Tyr103, Tyr104, Tyr105 of a heavy chain sequence of |
| | SEQ ID NO: 26 or Gly28, Ser29, Lys30, Tyr31, Tyr48, Asp50, Asn51, |
| | Glu52, Asn65, and Trp92 of a light chain sequence of SEQ ID NO: 27; |
| | An anti-TSLP antibody that specifically binds an epitope in human |
| | TSLP, wherein the epitope comprises at least one of the following |
| | residues: Lys38, Ala41, Leu44, Ser45, Thr46, Ser48, Lys49, Ile52, |
| | Thr53, Ser56, Gly57, Thr58, Lys59, Lys101, Gln145, and Arg149 of |
| | SEQ ID NO: 30; |
| WO2016/142426 | An anti-TSLP antibody comprising the amino acid sequence of SEQ ID |
| | NO: 31; |
| | An anti-TSLP antibody comprising a CDR1 comprising the sequence of |
| | SEQ ID NO: 32; a CDR2 comprising the sequence of SEQ ID NO: 33, |
| | and a CDR3 comprising the sequence of SEQ ID NO: 34; |
| | An anti-TSLP antibody comprising a CDR1 comprising the sequence of |
| | SEQ ID NO: 32; a CDR2 comprising the sequence of SEQ ID NO: 35, |
| | and a CDR3 comprising the sequence of SEQ ID NO: 34; |
| | An anti-TSLP antibody comprising a variant of the CDR1 of SEQ ID NO: |
| | 31 wherein the residue corresponding to residue 28 in SEQ ID NO: 31 is |
| | Pro, the residue corresponding to residue 30 in SEQ ID NO: 31 is Arg, |
| | the residue corresponding to residue 31 in SEQ ID NO: 31 is Asn, the |
| | residue corresponding to residue 32 in SEQ ID NO: 31 is Trp and the |
| | residue corresponding to residue 34 in SEQ ID NO: 31 is Asp; |
| | An anti-TSLP antibody comprising a variant of the CDR2 of SEQ ID NO: |
| | 31 wherein the residue corresponding to residue 50 in SEQ ID NO: 31 is |
| | Gly, the residue corresponding to residue 53 in SEQ ID NO: 31 is His |
| | and the residue corresponding to residue 55 in SEQ ID NO: 31 is Gln; |
| | An anti-TSLP antibody comprising a variant of the CDR3 of SEQ ID NO: |
| | 31 wherein the residue corresponding to residue 91 in SEQ ID NO: 31 is |
| | He, Leu, Val or Phe, the residue corresponding to residue 92 in SEQ ID |
| | NO: 31 is Gly or Ala, the residue corresponding to residue 93 in SEQ ID |
| | NO: 31 is Glu, Phe, Asp or Ser and the residue corresponding to residue |
| | 94 in SEQ ID NO: 31 is Asp. |
| WO2010/017468 | An anti-TSLP antibody (9B7) comprising a HC CDR3 comprising the |
| | sequence of SEQ ID NO: 38, wherein the other CDRs of the HC and LC |
| | comprise the sequences of SEQ ID NOs: 36, 37, and 39-41; |
| | An anti-TSLP antibody (6C5) comprising a HC CDR3 comprising the |
| | sequence of SEQ ID NO: 44, wherein the other CDRs of the HC and LC |
| | comprise the sequences of SEQ ID NOs: 42, 43, and 45-47; |
| | An anti-TSLP antibody (6A3) comprising a HC CDR3 comprising the |
| | sequence of SEQ ID NO: 50, wherein the other CDRs of the HC and LC |
| | comprise the sequences of SEQ ID NOs: 48, 49, and 51-53; |
| | An anti-TSLP antibody (1A11) comprising a HC CDR3 comprising the |
| | sequence of SEQ ID NO: 56, wherein the other CDRs of the HC and LC |
| | comprise the sequences of SEQ ID NOs: 54, 55, and 57-59; |
| | An anti-TSLP antibody comprising (i) heavy chain variable region of |
| | SEQ ID NO: 60 and/or the light chain variable region of SEQ ID NO: 61; |
| | An anti-TSLP antibody comprising (i) heavy chain variable region of |
| | SEQ ID NO: 62 and/or the light chain variable region of SEQ ID NO: 63; |
| | An anti-TSLP antibody comprising (i) heavy chain variable region of |
| | SEQ ID NO: 64 and/or the light chain variable region of SEQ ID NO: 65; |
| | An anti-TSLP antibody comprising (i) heavy chain variable region of |
| | SEQ ID NO: 66 and/or the light chain variable region of SEQ ID NO: 67; |
| | An anti-TSLP antibody comprising (i) heavy chain variable region of |
| | SEQ ID NO: 68 and/or the light chain variable region of SEQ ID NO: 69; |
| | An anti-TSLP antibody comprising a HC CDR selected from the group |
| | consisting of SEQ ID NO: 38, SEQ ID NO: 44, SEQ ID NO: 50 and SEQ |
| | ID NO: 56, and analogs thereof; |
| | An anti-TSLP antibody comprising a heavy chain comprising the |
| | following CDRs or analogs thereof CDRH1: RYNVH (SEQ ID NO: 36), |
| | CDRH2: MIWDGGSTDYNSALKS (SEQ ID NO: 37), CDRH3: NRYESG |
| | (SEQ ID NO: 38), and a light chain comprising the following CDRs or |
| | analogs thereof CDRL1: KSSQSLLNSGNRKNYLT (SEQ ID NO: 39), |
| | CDRL2: WASTRES (SEQ ID NO: 40), and CDRL3: QNDYTYPFTFGS |
| | (SEQ ID NO: 41); or |
| | An anti-TSLP antibody comprising a heavy chain comprising the |
| | following CDRs or analogs thereof CRDH1: AYWMS (SEQ ID |
| | NO: 42), CDRH2: EINPDSSTINCTPSLKD (SEQ ID NO: 43), CDRH3: |
| | RLRPFWYFDVW (SEQ ID NO: 44), and a light chain comprising the |
| | following CDRs or analogs thereof CDRL1: RSSQSIVQSNGNTYLE |
| | (SEQ ID NO: 45), CDRL2: KVSNRFS (SEQ ID NO: 46), and CDRL3: |
| | FQGSHVPRT (SEQ ID NO: 47); |
| | An anti-TSLP antibody comprising a heavy chain comprising the |
| | following CDRs or analogs thereof CRDH1: TDYAWN (SEQ ID NO: 48), |
| | CDRH2: YIFYSGSTTYTPSLKS (SEQ ID NO: 49), CDRH3: GGYDVNYF |
| | (SEQ ID NO: 50), and a light chain comprising the following CDRs or |
| | analogs thereof CDRL1: LASQTIGAWLA (SEQ ID NO: 51), CDRL2: |
| | AATRLAD (SEQ ID NO: 52), and CDRL3: QQFFSTPWT (SEQ ID |
| | NO: 53); |
| | An anti-TSLP antibody comprising a heavy chain comprising the |
| | following CDRs or analogs thereof CDRH1: GYTMN (SEQ ID NO: 54), |
| | CDRH2: LINPYNGVTSYNQKFK (SEQ ID NO: 55), CDRH3: |
| | GDGNYWYF (SEQ ID NO: 56), and a light chain comprising the |
| | following CDRs or analogs thereof CDRL1: SASSSVTYMHW (SEQ ID |
| | NO: 57), CDRL2: EISKLAS (SEQ ID NO: 58), and CDRL3: QEWNYPYTF |
| | (SEQ ID NO: 59); |
| | An anti-TSLP antibody comprising a HC CDR1 comprising the sequence |
| | of SEQ ID NO: 70; a CDR2 comprising the sequence of SEQ ID NO: 71, |
| | and a CDR3 comprising the sequence of SEQ ID NO: 72; |
| | An anti-TSLP antibody comprising a LC CDR1 comprising the sequence |
| | of SEQ ID NO: 73; a CDR2 comprising the sequence of SEQ ID NO: 74, |
| | and a CDR3 comprising the sequence of SEQ ID NO: 75; |
| US2012/0020988 | An anti-TSLP antibody comprising a heavy chain variable domain |
| | comprising a CDR1 region of SEQ ID NO: 76, a CDR2 region of SEQ ID |
| | NO: 77, and CDR3 region of SEQ ID NO: 78, and a light chain variable |
| | domain comprising a CDR1 region of SEQ ID NO: 79, a CDR2 region of |
| | SEQ ID NO: 80, and a CDR3 region of SEQ ID NO: 81. |
| | An anti-TSLP antibody comprising a heavy chain variable domain |
| | comprising SEQ ID NO: 82 and a light chain variable domain comprising |
| | SEQ ID NO: 83; |
| | An anti-TSLP antibody comprising a heavy chain variable domain |
| | comprising a CDR1 region of SEQ ID NO: 76 or 84, a CDR2 region of |
| | SEQ ID NO: 77 or 85, and CDR3 region of SEQ ID NO: 78, and a light |
| | chain variable domain comprising a CDR1 region of SEQ ID NO: 79 or |
| | 86, a CDR2 region of SEQ ID NO: 80, 87, or 88, and a CDR3 region of |
| | SEQ ID NO: 81. |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 87 and a CDR3 region of SEQ ID NO: 81; |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 88 and a CDR3 region of SEQ ID NO: 81; |
| | An anti-TSLP antibody comprising a heavy chain variable domain |
| | comprising a CDR1 region of SEQ ID NO: 84, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 88 and a CDR3 region of SEQ ID NO: 81; or |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 80 and a CDR3 region of SEQ ID NO: 81. |
| | An anti-TSLP antibody comprising a heavy chain variable domain |
| | comprises SEQ ID NO: 89 and a light chain variable domain comprises |
| | SEQ ID NO: 90; |
| | An anti-TSLP antibody comprising a heavy chain variable domain |
| | comprises SEQ ID NO: 89 and a light chain variable domain comprises |
| | SEQ ID NO: 91; |
| | An anti-TSLP antibody comprising a heavy chain variable domain |
| | comprises SEQ ID NO: 92 and a light chain variable domain comprises |
| | SEQ ID NO: 93; |
| | An anti-TSLP antibody comprising a heavy chain variable domain |
| | comprises SEQ ID NO: 89 and a light chain variable domain comprises |
| | SEQ ID NO: 94, |
| US8637019 | An anti-TSLP antibody comprising heavy chain variable region |
| | comprising: a CDR-H1 sequence comprising SEQ ID NO: 95, a CDR-H2 |
| | sequence comprising SEQ ID NO: 96, and a CDR-H3 sequence |
| | comprising SEQ ID NO: 97; and/or an antibody light chain variable |
| | region or a TSLP-binding fragment thereof, said light chain variable |
| | region comprising: a CDR-L1 sequence comprising SEQ ID NO: 98, a |
| | CDR-L2 sequence comprising SEQ ID NO: 99, and a CDR-L3 sequence |
| | comprising SEQ ID NO: 100. |
| | An anti-TSLP antibody comprising a heavy chain variable region |
| | comprises the amino acid sequence of SEQ ID NO: 101 and the light |
| | chain variable region comprises the amino acid sequence of SEQ ID |
| | NO: 102. |
| | An anti-TSLP antibody comprising SEQ ID NO: 103 and SEQ ID |
| | NO: 104. |

It is provided that the subject to be treated is human. The subject may be an adult, an adolescent or a child.

Therapeutic antibody (or antibody variant) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, bimonthly, or at a longer interval.

In various embodiments, the amounts of therapeutic agent, such as a bivalent antibody having two TSLP binding sites, in a given dosage may vary according to the size of the individual to whom the therapy is being administered as well as the characteristics of the disorder being treated.

In exemplary treatments, the anti-TSLP antibody or antibody variant is administered in a dose range of about 140 mg to about 420 mg per dose. In various embodiments, the dose may be given in about 140 mg, 210 mg, 280 mg or 420 mg. In various embodiments, the anti-TSLP antibody or antibody variant may be administered at a dose of about 140, 150, 160, 170, 180, 190, 200, 210, 220, 230, 240, 250, 260, 270, 280, 290, 300, 310, 320, 330, 340, 350, 360, 370, 380, 390, 400, 410 or 420 mg per dose. These concentrations may be administered as a single dosage form or as multiple doses. The above doses are given every two weeks or every four weeks. In various embodiments, the anti-TSLP antibody or antibody variant is administered at a single dose of 210 mg every two weeks or every four weeks. In various embodiments, the anti-TSLP antibody or antibody variant is administered at a single dose of 210 mg every four weeks.

For antibody variants, the amount of antibody variant should be such that the number of TSLP binding sites that are in the dose have an equimolar number of TSLP binding sites to canonical bivalent antibody described above.

It is provided that the anti-TSLP antibody or antibody variant is administered every 2 weeks or every 4 weeks for a period of at least 4 months, 6 months, 9 months, 1 year or more. In various embodiments, the administration is subcutaneous or intravenous.

Treatment with the anti-TSLP antibody or antibody variant is provided to improve one or more of the following measures of chronic rhinosinusitis (with or without nasal polyps): Nasal Polyp Score (NSP), Nasal Congestion Score (NCS), loss of smell, SinNasal Outcome Test 22 Item (SNOT-22), EuroQOL quality of life 5-dimensions 3-level version (EQ-5D-3L), Asthma Control Questionnaire (ACQ-6), Lund-Mackay score, modified Lund-Mackay Score, incidence of nasal polyp surgery and/or systemic corticosteroids (SCS).

In one embodiment, the administration improves one or more symptoms of chronic rhinosinusitis including nasal blockage, nasal congestion, runny nose, post-nasal drip, mucus drainage down the throat, prominent nasal obstruction, nasal discharge, loss of smell, headache, facial pain, facial pressure, difficulty with sense of smell, asthma symptoms, difficulty with sleeping due to nasal symptoms, difficulty with daily activities due to nasal symptoms, optionally as measured by chronic rhinosinusitis patient electronic diary.

It is contemplated that the improvement in score is related to the minimal clinically important differences (MCID) in symptom measurement for a patient treated according to a method herein, or to the least square mean difference compared to placebo group. MCID refers to the minimal improvement considered therapeutically relevant by a patient or physician. See e.g., Cook et al., J Man Manip Ther. 2008; 16 (4): E82-E83; Phillips et al., Clin Otolaryngol 2018 43 (5): 1328-1334.

For example, a clinically relevant improvement in nasal congestion score (NC) includes a MCID of between 0.5 to 1.2, e.g., 0.5, 0.6, 0.7, 0.8, 0.89, 0.9, 1.0, 1.1, or 1.2 or more compared to baseline or untreated control levels. A clinically relevant improvement in nasal polyp score (NPS) includes a MCID of between 1 and 3, e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 compared to baseline or untreated control levels. A clinically relevant improvement includes a reduction in treatment with systemic corticosteroids and/or surgery with a MCID of between 60% to 90%, e.g., 60%, 65%, 70%, 75%, 80%, 85%, 90% compared to baseline or untreated control levels. A clinically relevant improvement includes a reduction in requiring nasal surgery with a MCID of between 60% to 90%, e.g., 60%, 65%, 70%, 75%, 80%, 85%, 90% compared to baseline or untreated control levels. A clinically relevant improvement in Lund Mackey score (NC) includes an MCID of between 5 and 10, e.g., 5, 6, 7, 8, 9 or 10 compared to baseline or untreated control levels. A clinically relevant improvement in sinonasal symptoms (SNOT-22) includes a MCID of between 15 and 30, e.g., 15, 16, 17, 18, 19, 20, 21, 22, 23, 24, 25, 26, 27, 28, 29 or 30, compared to baseline or untreated control levels. Comparisons may be done throughout the treatment period, for example at week 28 or week 52 or more.

In various embodiments, the NPS or NCS score is reduced by one, two or three points. In various embodiments, the subject has an NPS of 0 (none) or 1 (mild) at week 52 after treatment.

In various embodiments, treatment with anti-TSLP modulates the levels of one or more biomarkers of chronic rhinosinusitis, including, cytokines, IgE, CCL17, CCL18, CCL22, and RNA transcriptional changes in nasal epithelium. In various embodiments, treatment with anti-TSLP reduces the level of Th2 cytokines. In various embodiments, the treatment modulates levels of or activity of IL-4, IL-5, IL-13, IL-17, IL-22, IL-23, IL-31, or combinations thereof.

The treatment also improves one or more symptoms of chronic rhinosinusitis as measured by a Nasal Polyposis Symptom Diary (NPSD) and Nasal Polyposis Symptom Screening Assessment (NPSSA). Symptoms include nasal blockage, nasal congestion, runny nose, postnasal drip (mucus drainage down the throat), headache, facial pain, facial pressure, and difficulty with sense of smell) and symptom impacts (difficulty with sleeping due to nasal symptoms and difficulty with daily activities due to nasal symptoms); mobility, self-care, usual activities, pain/discomfort, and anxiety/depression measured by the EuroQOL quality of life 5-dimensions 3-level version (EQ-5D-3L).

In various embodiments, treatment with the anti-TSLP antibody delays the time to an chronic rhinosinusitis exacerbation or flare up compared to a subject not receiving the anti-TSLP antibody.

Also provided in the present disclosure is the administration of multiple agents, such as an antibody composition in conjunction with a second agent as described herein, including but not limited to an anti-inflammatory agent or asthma therapy.

However, it is provided that, in various embodiments, the administration reduces frequency of or levels of co-administered therapy in the subject. Exemplary co-administered therapies include, but are not limited to, systemic corticosteroids, dupilumab, immunosuppressive or immunomodulating drugs (e.g., cyclosporine, mycophenolate-mofetil, interferon (IFN)-gamma, Janus kinase inhibitors, azathioprine, methotrexate), anti-IL-13 antibodies, anti-IL-5 pathway antibodies (benralizumab, mepolizumab, reslizumab), or combinations thereof. In various embodiments, the administration reduces or eliminates the need for corticosteroid therapy or other adjunct therapy.

### Formulations

In some embodiments, the disclosure provides use of pharmaceutical compositions comprising a therapeutically effective amount of an anti-TSLP antibody or antibody variant together with a pharmaceutically acceptable diluent, carrier, solubilizer, emulsifier, preservative, and/or adjuvant. In addition, the disclosure provides methods of treating a subject by administering such pharmaceutical composition.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

A suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor.

The formulation components are present preferably in concentrations that are acceptable to the site of administration. In certain embodiments, buffers are used to maintain the composition at physiological pH or at a slightly lower pH, typically within a pH range of from about 4.5 to about 8. Including about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0.

In various embodiments, the anti-TSLP antibody or antibody variant is in a formulation containing one or more basic amino acids (e.g., arginine, histidine or lysine) or salt thereof, or a calcium or magnesium salt, and a surfactant. In various embodiments, the formulation comprises 0.005% (w/v) to about 0.015% (w/v) polysorbate 20 or polysorbate 80. In various embodiments, the formulation is at pH between 4.5 and 6.8. In various embodiments, the antibody or antibody fragment in the formulation is at a concentration of greater than 110 mg/ml, e.g., from about 110 mg/ml to about 250 mg/ml, e.g., from about 140 mg/ml to about 250 mg/ml, from about 160 mg/mL to about 250 mg/mL, or from about 140 mg/mL to about 210 mg/mL, or about 180 mg/ml or about 210 mg/ml. The formulation may be stored at 2° to 8° C. or −20° to −70° C. Exemplary formulations are described in co-owned applications PCT/US2021/018561 and PCT/US2021/17880.

When parenteral administration is carried out, the therapeutic compositions for use may be provided in the form of a pyrogen-free, parenterally acceptable aqueous solution comprising the desired anti-TSLP antibody in a pharmaceutically acceptable vehicle. A particularly suitable vehicle for parenteral injection is sterile distilled water in which the antibody is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the antibody.

### EXAMPLES

### Example 1—A Phase 3 Study to Evaluate the Effect of Tezepelumab in Chronic Rhinosinusitis with Nasal Polyps (CRSwNP)

The aim of the present study is to investigate the efficacy and safety of tezepelumab in participants with severe chronic rhinosinusitis with nasal polyps (CRSwNP), with or without co-morbid asthma/AERD/NSAID-ERD, whose severity is consistent with a need for surgery (total nasal polyps score (NPS) ≥5 (at least 2 for each nostril) despite documented treatment with systemic corticosteroid (SCS) or prior surgery for NP. The effect of tezepelumab 210 mg every 4 weeks (Q4W) on nasal polyps is assessed on top of standard of care therapy with intranasal corticosteroids (INCS) over a 52-week treatment period and up to 24-week post-treatment follow up period.

Approximately, 400 participants are randomized globally 1:1 to receive tezepelumab 210 mg or matching placebo.

Participants are stratified by region, prior nasal polyp surgery and co-morbid asthma/aspirin-induced respiratory disease (AERD)/nonsteroidal anti-inflammatory drug exacerbated respiratory disease (NSAID-ERD). Randomization will be monitored to ensure that 50%-70% of the study population will have co-morbid asthma/AERD/NSAID-ERD, and at least 50% will have had prior surgery for CRSwNP.

After enrolment, eligible participants receive a standardized dose of Intranasal Mometasone Furoate Nasal Spray (MFNS) or equivalent INCS and will enter a 5-week screening/run in period. Equivalent dose should refer to the highest approved country INCS dose for CRSwNP. Participants who meet eligibility criteria will be randomized at Week 0 (Day 0) to receive either placebo or tezepelumab 210 mg SC starting at randomization (Week 0) and then every 4 weeks thereafter. An end of treatment (EOT) visit will be conducted at Week 52.

There will be two different follow-up periods after the 52-week treatment period. Approximately, the first 200 participants that complete the 52-week treatment period will have a 24-week follow-up (FU) period without any investigation product (IP) (tezepelumab). The last remaining 200 participants will have a 12-week FU period without any IP.

### Endpoints and Assessments

Primary Endpoints are shown in Table 1.

TABLE 1
Primary Endpoints

| | | |
|---|---|---|
| NPS at Week | H01a: Difference in mean change | H11a: Difference in mean change from |
| 52 | from baseline in NPS at 52 weeks | baseline in NPS at 52 weeks |
| | (tezepelumab minus placebo) = 0 | (tezepelumab minus placebo) ≠ 0 |
| NCS at Week | H01b: Difference in mean change | H11b: Difference in mean change from |
| 52 | from baseline in NCS at 52 weeks | baseline in NCS at 52 weeks |
| | (tezepelumab minus placebo) = 0 | (tezepelumab minus placebo) ≠ 0 |

Nasal Polyp Score (NPS): The NPS is the sum of the right and left nostril scores (maximum 8), as evaluated by nasal endoscopy. Total NPS is graded based on polyp size described in Table 2. Nasal endoscopy may be preceded by local administration of anaesthetic drugs in combination with a decongestant, as per local medical practice.

TABLE 2
Endoscopic NPS Within each Nostril

| Polyp score | Polyp size |
|---|---|
| 0 | No polyps |
| 1 | Small polyps in the middle meatus not |
| | reaching below the inferior border of |
| | the middle turbinate |
| 2 | Polyps reaching below the lower |
| | border of the middle turbinate |
| 3 | Polyps reaching the lower border of the |
| | inferior turbinate or a middle meatal |
| | polyp with a score of 2 with any additional |
| | polyp medial to the middle turbinate |
| 4 | Large polyps causing complete or near- |
| | complete obstruction of the inferior |
| | nasal cavity i.e. touching the floor of the nose |

Secondary Endpoints include those shown in Table 3.

TABLE 3
Secondary Endpoints

| | | |
|---|---|---|
| Loss of smell | H02a: Difference in mean change | H12a: Difference in mean change from |
| at Week 52 | from baseline in loss of smell at 52 | baseline in loss of smell at 52 weeks |
| | weeks (tezepelumab minus placebo) = 0 | (tezepelumab minus placebo) ≠ 0 |
| SNOT-22 at | H02b: Difference in mean change | H12b: Difference in mean change from |
| Week 52 | from baseline in SNOT-22 at 52 | baseline in SNOT-22 at 52 weeks |
| | weeks (tezepelumab minus placebo) = 0 | (tezepelumab minus placebo) ≠ 0 |
| LMK at Week | H02c: Difference in mean change | H12c: Difference in mean change from |
| 52 | from baseline in LMK at 52 weeks | baseline in LMK score at 52 weeks |
| | (tezepelumab minus placebo) = 0 | (tezepelumab minus placebo) ≠ 0 |
| Time to SCS | H02d: HR of time to first SCS and/or | H12d: HR of time to first SCS and/or |
| and/or surgery | surgery decision | surgery decision (tezepelumab/placebo) ≠ 1 |
| decision up to | (tezepelumab/placebo) = 1 | |
| Week 52 | | |
| Time to | H03: HR of time to first surgery | H13: HR of time to first surgery decision |
| surgery | decision (tezepelumab/placebo) = 1 | (tezepelumab/placebo) ≠ 1 |
| decision up to | | |
| Week 52 | | |
| Time to SCS | H04: HR of time to first SCS | H14: HR of time to first SCS |
| up to Week 52 | (tezepelumab/placebo) = 1 | (tezepelumab/placebo) ≠ 1 |
| NPSD TSS | H05: Difference in mean change from | H15: Difference in mean change from |
| | baseline in NPSD TSS at 52 weeks | baseline in NPSD TSS at 52 weeks |
| | (tezepelumab minus placebo) = 0 | (tezepelumab minus placebo) ≠ 0 |
| Maximum NPS | H06: OR for proportion of participants | H16: OR for proportion of participants |
| of 1 (NPS ≤ 1) | with a maximum NPS of 1 in each | with a maximum NPS of 1 in each nostril |
| in each nostril | nostril at 52 weeks | at 52 weeks (tezepelumab/placebo) ≠ 1 |
| at Week 52 | (tezepelumab/placebo) = 1 | |
| Maximum NPS | H07: OR for proportion of participants | H17: OR for proportion of participants |
| of 1 (NPS ≤ 1) | with a maximum NPS of 1 in each | with a maximum NPS of 1 in each nostril |
| in each nostril | nostril and NPSD TSS response at 52 | and NPSD TSS response at 52 weeks |
| and NPSD | weeks (tezepelumab/placebo) = 1 | (tezepelumab/placebo) ≠ 1 |
| TSS response | | |
| at Week 52 | | |
| FEV₁ at Week | H08: Difference in mean change from | H18: Difference in mean change from |
| 52 | baseline in pre-BD FEV₁ at 52 weeks | baseline in pre-BD FEV₁ at 52 weeks |
| Subset of | (tezepelumab minus placebo) = 0 | (tezepelumab minus placebo) ≠ 0 |
| participants | | |
| with co-morbid | | |
| asthma/AERD/ | | |
| NSAID-ERD | | |

BD bronchodilator;
FEV_{1 forced} exhalation volume in 1 second;
LMK Lund Mackay score;
NPS nasal polyp score;
NCS nasal congestion score;
SCS systemic corticosteroids;
SNOT-22 SinoNasal outcome test

Clinical Outcome Assessments. Participants complete all patient-reported outcomes assessments (PRO) using a handheld ePRO device. The ePRO device is used to capture symptoms (Nasal Polyposis Symptom Screening Assessment) and health-related quality of life [(HRQOL) SNOT-22] screening data at Visit 1.

Participant compliance should be checked weekly (at minimum) to ensure that the participant is completing the assessments as scheduled. Monitoring of participant adherence to the diary is important during the baseline period (Study Day-13 to Day 0) to ensure that the participant meets applicable criteria for randomization.

Nasal Polyposis Symptom Diary (NPSD) and Nasal Polyposis Symptom Screening Assessment (NPSSA): The participant will complete an 11-item NP symptom diary (NPSD) each morning throughout the screening, treatment, and follow-up periods. The participant is asked to consider their experience with NP/nasal polyps over the past 24 hours when responding to each question. Participants are asked to report their experience with NP symptoms (nasal blockage, nasal congestion, runny nose, postnasal drip (mucus drainage down the throat), headache, facial pain, facial pressure, and difficulty with sense of smell) and symptom impacts (difficulty with sleeping due to nasal symptoms and difficulty with daily activities due to nasal symptoms). Participants report the severity of each symptom and symptom impact at its worst using a 4-point verbal rating scale (0-None to 3-Severe). A total symptom score (TSS) is calculated by taking the sum of the 8 equally weighted symptom items. A single item to capture INCS compliance (yes or no) will be administered after the symptom and symptom impact item.

Nasal Congestion Score: NC score (NCS) is captured by an item in the NPSD asking participants to rate the severity of their worst nasal congestion over the past 24 hours using the following response options: 0-None; 1-Mild; 2-Moderate; 3-Severe. Baseline will be the mean of daily responses from Day-13 to Day 0. Bi-weekly (14-day) mean NCS will be calculated if at least 8 days in each 14-day period has evaluable data.

Loss of Smell: Loss of smell is captured by an item in the NPSD asking participants to rate the severity of their worst difficulty with sense of smell over the past 24 hours using the following response options: 0-None; 1-Mild; 2-Moderate; 3-Severe. Baseline will be the mean of daily responses from Day-13 to Day 0. Bi-weekly (14-day) mean loss of smell will be calculated if at least 8 days in each 14-day period has evaluable data.

SinoNasal Outcome Test 22 item: The SNOT-22 is a condition-specific HRQOL assessment which captures participant-reported physical problems, functional limitations, and emotional consequences of SinoNasal conditions (Piccirillo et al 2002; Hopkins et al 2009). Patient-reported symptom severity and symptom impact over the past 2 weeks are captured via a 6-point scale (0-No Problem to 5-Problem as bad as it can be). The total score is the sum of item scores and has a range from 0 to 110 (higher scores indicate poorer outcomes). A Minimal Clinical Importance Difference (MCID) of 8.90 has been established for individual score change (Hopkins et al 2009).

The SNOT-22 test can be divided further into domains to provide a domain score. Domains and the factors that are considered in the domain score include: Nasal domain: need to blow nose, nasal blockage, sneezing, runny nose, cough, post-nasal discharge, thick nasal discharge, decreased sense of smell/taste; Ear/Facial Domain: ear fullness, dizziness, ear pain, facial pain/pressure; Sleep Domain: difficulty falling asleep, wake up at night, lack of a good night's sleep, wake up tired; Function Domain: fatigue, reduced productivity, reduced concentration; Emotion Domain: frustrated/restless/irritable, sad, embarrassed (Khan et al., Laryngoscope. 2022 132 (5): 933-941). Each domain is scored on a 0-5 scale, with 0 being the lowest.

Short-Form 36-item Health survey, version 2: The Short Form 36-item Health survey, Version 2 (standard recall) (SF-36v2) is a 36-item, wself-report survey of functional health and well-being, with a 4-week recall period (QualityMetric 2011). Responses to 35 of the 36 items are used to compute an 8-domain profile of functional health and well-being scores. The remaining item, referred to as the ‘Health Transition’ item, asks participants to rate how their current state of health compared to their state of health 1 year ago, and is not used to calculate domain scores. The 8-domain profile consists of the following subscales: Physical Functioning (PF), Role Limitations due to Physical Health (RP), Bodily Pain (BP), General Health Perceptions (GH), Vitality (VT), Social Functioning (SF), Role Limitations due to Emotional Problems (RE), and Mental Health (MH). Psychometrically-based physical and mental health component summary scores (PCS and MCS, respectively) are computed from subscale scores to give a broader metric of physical and mental HRQOL.

Two types of thresholds have been developed for interpretation of SF-36v2 scores. The first type is suitable for comparing group mean scores and is generally referred to as the MCID. The second type is suitable for interpreting change at the individual level and is referred to as the responder threshold or responder definition (QualityMetric 2011).

TABLE 4
Threshold Values for the SF-36v2 Scal4e and Summary Measures

| | SF-36v2 score | | | | | | | | | |
|---|---|---|---|---|---|---|---|---|---|---|
| Threshold | PCS | MCS | PF | RP | BP | GH | VT | SF | RE | MH |
| Group difference | 2 | 3 | 3 | 3 | 3 | 2 | 2 | 3 | 4 | 3 |
| Individual change | 3.4 | 4.6 | 4.3 | 3.4 | 6.2 | 7.2 | 6.2 | 6.9 | 4.5 | 6.2 |

BP Bodily Pain;
GH General Health Perceptions;
MCS mental health component summary;
MH Mental Health;
PCS physical component summary;
PF Physical Functioning;
RE Emotional Problems;
RP Role Limitations due to Physical Health;
SF Social Functioning;
VT Vitality

Asthma Control Questionnaire (for asthma/AERD/NSAID-ERD participants only): The Asthma Control Questionnaire (ACQ-6) is an assessment of asthma symptoms (night-time waking, symptoms on waking, activity limitation, shortness of breath, wheezing, and short acting beta-agonist use). Participants are asked to recall their level of asthma control during the previous week by responding to one bronchodilator use question and 5 symptom questions. Questions are weighted equally and scored from 0 (totally controlled) to 6 (severely uncontrolled). The mean ACQ-6 score is the mean of the responses. Mean scores of ≤0.75 indicate well-controlled asthma, scores between 0.75 and <1.5 indicate partly controlled asthma, and a score ≥1.5 indicates not well controlled asthma (Juniper et al 2006). Individual changes of at least 0.5 are considered to be clinically meaningful.

University of Pennsylvania Smell Identification Test: The University of Pennsylvania Smell Identification Test (UPSIT) is a quantitative test of olfactory function which uses microencapsulated odorants that are released by scratching standardized odour-impregnated test booklets (Doty et al 1984). Four booklets each with 10 odorants each are used for the test. Participants are asked to identify the odor using multiple choice format which lists different possibilities. The test is forced-choice, i.e., the participant is required to mark one of the four alternatives even if no smell is perceived. Scores are based on number of correctly identified odors (score range 0 to 40).

Patient Global Impression of Severity and Change: The Patient Global Impression of Severity (PGI-S) is a single item designed to capture the participant's perception of overall NP symptom severity at the time of completion using a 6-point categorical response scale (0—No Symptoms to 5—Very Severe). The Patient Global Impression of Change (PGI-C) instrument captures the participant's overall evaluation of response to treatment since first dose of IP. The participant is asked to report the degree to which their health status has changed using a 7-point scale (1—Much Better to 7—Much Worse).

Work Productivity and Activity Impairment (WPAI) Questionnaire: The Work Productivity and Activity Impairment questionnaire (WPAI, General Health version 2.0) is a self-administered tool comprised of 6 questions which address absenteeism, presenteeism (reduced effectiveness while working), overall work productivity loss (absenteeism plus presenteeism), and activity impairment. This validated tool captures data from the past 7 days. WPAI outcomes are scored as impairment percentages, with a higher percentage indicating greater impairment and less productivity (Reilly et al 1993).

Sinus Computed Tomography: Computed tomography (CT) will be performed in all participants. Referral for baseline CT Scan should be provided at V2 (Week-2) and must be performed after nasal biopsy procedure (if applicable) at V2 only if the participant has met all eligibility criteria at V2 including NPS score confirmation by central readers. At V17 (EOT/IPD), the CT Scan should be performed before the nasal biopsy procedure is completed for participants in the nasal biopsy sub-study.

For participants who undergo or are planned for surgery for NP during the treatment period, a CT scan should be performed prior to the first NP surgery per the IPD visit. For participants who require SCS for the treatment of NP, a CT scan should be performed prior to the first course of SCS, if possible, and at Week 52. For participants who receive multiple NP surgeries and/or SCS treatments for NP, the CT Scan should be performed prior to the first occurrence of either NP Surgery or SCS treatment for NP. For participants who discontinue IP for other reasons, a CT scan should be performed at the IPD visit as well as at Week 52, if participant opts for follow up option 1 or 2. Sinus CT images are used to derive Lund-Mackay scores (LMSs) and Zinreich (modified Lund-Mackay) scores based on the visual assessment by independent central readers, and for quantitative estimation of a sinus severity score which is representative of sinus disease burden.

Lund-Mackay score: The Lund-Mackay score scoring system is used to provide a semi-quantitative assessment of nasal sinuses on sinus CT scans (Lund and Mackay 1993). Based on the sinus CT images, the five sinuses (maxillary, anterior ethmoid, posterior ethmoid, sphenoid and frontal) on each side are scored by central radiologist as follows:

TABLE 5
Lund-Mackay Score

| Score | CT scan assessment |
|---|---|
| 0 | No abnormality |
| 1 | Partial opacification |
| 2 | Total opacification |

The ostiomeatal complex is scored for right and left sides.

TABLE 6
Ostiomeatal Complex Score

| Score | CT scan assessment |
|---|---|
| 0 | Not occluded |
| 2 | Occluded |

The maximum total score is 24.

Quantitative measurement of sinus disease burden on sinus computed tomography: Quantitative assessment of sinus CT image data will be used to derive an objective measure of sinus disease burden called sinus severity score (Pallanch et al 2013). This is defined as:

Sinus severity score=sinus mucosal volume/(sinus mucosal volume+sinus air volume)*100%.

The following parameters used to calculate the sinus severity score: (a) sinus air volume (mL); (b) sinus mucosal volume (mL). Image analysis is performed centrally.

Zinreich (modified Lund Mackay) score: In addition to the Lund Mackay scoring described above, the same CT images are also scored using the modified Lund Mackay (Zinreich) scoring system (Okushi et al 2013, Likness et al 2014). All five sinuses (maxillary, anterior ethmoid, posterior ethmoid, sphenoid and frontal) on each side will be scored based on the percentage of opacification from mucosal thickening according to Table 7.

TABLE 7
Modified Lund Mackay (Zinreich) Score

| Score | Percent opacification |
|---|---|
| 0 | 0% |
| 1 | 1%-25% |
| 2 | 26%-50% |
| 3 | 51%-75% |
| 4 | 76%-99% |
| 5 | 100% |

Post-randomization Nasal Polyp Surgery and/or SCS use: Participants who have a scheduled NP surgery at the time of the study enrolment and randomization should not be randomized (refer to exclusion criterion 15). After randomization, surgery should not be planned for the first 3 months unless emergent or deemed necessary by the physician.

Rescue treatment of NP or other reasons is defined as requiring treatment with systemic corticosteroids (SCS) for at least 3 consecutive days (a single depo-injectable dose of corticosteroids will be considered equivalent to a 3-day course of systemic corticosteroids). A course of SCS is considered continuous if treatment episodes are separated by less than 7 days.

If SCS is used for treatment of NP for ≥3 consecutive days, a CT scan and a nasal endoscopy assessment of NPS should be performed prior to first course of treatment with SCS (unless the subject has already had a CT scan prior to a NP surgery post randomization).

If SCS use for treatment of any other reason than NP is close to V10 (Week 24) or V17 (Week 52), the visit should be rescheduled at least 2 weeks after the last dose of SCS.

Spirometry (asthma/AERD/NSAID-ERD participants only): Lung function (FEV1, FEF 25-75% and FVC) are measured by spirometry at the study site using equipment provided by a central vendor. The MasterScope is a system which will be used on-site to perform and record results for procedures including, but not limited to, spirometry, NPIF and FeNO. Spirometry is performed by the Investigator or authorized delegate according to American Thoracic Society/European Respiratory Society (ATS/ERS) guidelines or local guidelines (Graham et al 2019). The MasterScope kit will include a spirometer handle which will be used to record spirometry values at the site. The Global Lung Function Initiative (GLI) equations may be used to determine the Predicted Normal Values (PNV) and are pre-programmed into the spirometer (Quanjer et al 2012) FEV1, expressed as percent of the PNV, will be calculated as follows:

$$FEV 1 \% of PNV = ( FEV 1 measured / FEV 1 PNV ) \times 100$$

FeNO (asthma/AERD/NSAID-ERD participants only): Airway inflammation is evaluated using a standardized single-breath FeNO test in accordance with the SoA. A single exhalation technique recommended by the manufacturer will be followed (Alving et al 2017). Participants will be asked whether they have had a respiratory infection in the 2 weeks prior to the measurement. The FeNO measurements are performed within 2 weeks of a respiratory infection. The FeNO test is performed prior to spirometry. Participants should not use their rescue SABA medication (e.g., albuterol/salbutamol) within 6 hours of the measurement. Inhaled BDs (including ICS/LABA) should be withheld for the effect duration specific to the BD as described in the spirometry section.

The NIOX VERO® Airway Inflammation Monitor is used to measured FeNO.

Nasal Peak Inspiratory Flow: Nasal peak inspiratory flow (NPIF) evaluation represents a physiological measure of the air flow through both nasal cavities during forced inspiration expressed in liters per minute. Nasal inspiration correlates most with the participative feeling of obstruction and is the best validated technique for monitoring nasal flow in clinical trials. A minimum of three, up to a maximum of eight NPIF efforts will be performed by the participant; all values will be recorded by the participant on the MasterScope and the highest value will be used for evaluation.

The NPIF will be performed at enrollment/screening V1 (Week-5), V3 (Week 0), V7 (Week 12), V10 (Week 24), V13 (Week 36), V17 (Week 52), V19 (Week 64), V21 (Week 76).

The nasal flow is expressed in liters per minute, and consecutive measurements are performed. Taking the best of 3 outcomes with less than 10% variation is considered to be the best means of expression of the result (Scadding et al 2011).

Asthma exacerbations (for asthma/AERD/NSAID-ERD participants only): During the study, an asthma exacerbation will be defined as a worsening of asthma that leads to any of the following:
A temporary bolus/burst of systemic corticosteroids (or a temporary increase in stable OCS background dose) for at least 3 consecutive days to treat symptoms of asthma worsening; a single depo-injectable dose of corticosteroids will be considered equivalent to a 3-day burst of systemic corticosteroids.
An emergency room or urgent care visit (defined as evaluation and treatment for <24 hours in an emergency department or urgent care center) due to asthma that required systemic corticosteroids (as per the above).
An inpatient hospitalization (defined as admission to an inpatient facility and/or evaluation and treatment in a healthcare facility for ≥24 hours) due to asthma.

Participants are required to report any of the following in the eDiary:
An increase in rescue medication use of 4 or more puffs on at least 2 consecutive days compared with the average use during baseline or use of 12 puffs/day on any one day, and/or;
An additional nebulized 32 agonist use on at least 2 consecutive days compared with the average use during baseline, and/or;
An increase of 2 or more nights with awakenings due to asthma requiring rescue medication over a 7-day period compared with the average during baseline, and/or ≥6 out of previous 7 nights with awakenings due to asthma requiring rescue medication (this criterion should be met on 2 consecutive days).

If an exacerbation event is not associated with deterioration in at least 1 of the pre-specified objective measurements, the Investigator will have to justify the decision for defining the event as an exacerbation and record it in the eCRF. Events that are not supported by any objective assessment will be deemed not to be a protocol-defined exacerbation.

The start of an exacerbation is defined as the start date of systemic corticosteroids or of a temporary increase in a stable OCS background dose, date of ER or urgent care visits requiring systemic corticosteroids, or date of hospital admission due to asthma, whichever occurs earlier. The end date of an exacerbation is defined as the last date of systemic corticosteroids or of a temporary increase in a stable OCS background dose, date of ER or urgent care visit, or date of hospital discharge, whichever occurs later.

If less than 7 days have elapsed since the end date of an asthma exacerbation and the start date of a new asthma exacerbation, the second event will be considered a relapse of the prior asthma exacerbation

EQ-5D-3L. The EuroQOL quality of life 5-dimensions 3-level version (EQ-5D-3L) is a standardized instrument for use as a measure of health-related quality of life (HRQOL) and was developed by EuroQol (Brooks, 1996). It defines health in terms of 5 dimensions: mobility, self-care, usual activities, pain/discomfort, and anxiety/depression. Each dimension has 3 ordinal levels of severity: 1, no problem; 2, some problems; and 3, severe problems. Overall health state is defined as a 5-digit number. The participant will be asked to indicate his/her current health state by selecting the most appropriate level in each of the 5 dimensions. The questionnaire also includes a VAS, where the participant will be asked to rate current health status on a scale of 0 to 100, with 0 being the worst imaginable health state.

Patient Global Impression of Severity. The PGI-S is a single item designed to capture the subject's perception of overall symptom severity at the time of completion on a 5-point categorical response scale (no symptoms to very severe symptoms).

Adverse Events: All adverse events observed by the investigator or reported by the subject that occur after the first dose of investigational product through the end of study/safety follow-up visit or 20 weeks after the last administration of investigational product are to be collected/reported.

### Biomarkers

Serum Immunoglobulins. The levels of total immunoglobulin E (IgE) and an assessment for the presence of allergen-specific IgE (IgE FEIA) will be collected at the pre-specified scheduled visits prior to IP administration (pre-dose) according to the SoA and evaluated by a central laboratory.

Nasal epithelial lining fluid samples will be obtained at scheduled visits. Nasal epithelial lining fluid biomarkers will be measured to evaluate the pharmacology of tezepelumab and to evaluate changes in biomarkers related to CRSwNP, inflammation and the TSLP pathway. Baseline and early post-dose levels of nasal epithelial lining fluid biomarkers may also be used to explore for potential predictive biomarkers of response or exposure to tezepelumab. The specific biomarkers that may be analyzed include but are not limited to cytokines, chemokines and inflammatory mediators associated with CRSwNP and the TSLP pathway. Drug concentrations may also be measured in the nasal epithelial lining fluid samples, if feasible. If appropriate, urea concentrations may be measured to correct for the dilution factor of the nasal lining fluid samples.

In addition, a nasal biopsy sub-study will be conducted at selected sites, for up to approximately 60 participants in total. Nasal biopsies will be collected at V2 (Week-2) and EOT (Week 52) to evaluate the effect of tezepelumab on inflammatory cell infiltrate and gene expression by transcriptomic profiling. At V2, nasal biopsy should be performed after nasal epithelial lining fluid collection and before NPS assessment or CT Scan. If nasal biopsy sample cannot be collected at Visit 2, then participant should be withdrawn from nasal biopsy sub-study and can continue on the main study. In this event, NLF collection at Visit 2 and Staph A-enterotoxin specific IgE status sample collection at Visit 3 should be not performed. At EOT, nasal biopsy should be performed after NPS assessment, nasal epithelial lining fluid collection, and CT Scan. Collection of nasal biopsy sample may be postponed for up to 7 days after EOT visit, only if appropriate kit supplies are not available at the time of EOT visit.

Staphylococcus Aureus Characterization. Staphylococcus aureus (S. aureus; Staph A) enterotoxin IgE will only be performed in the nasal biopsy sub-study participants. Staph A bacterial culture from nasal swabs will be collected for all participants and evaluated by a central laboratory. These tests will be performed at the scheduled visits according to the SoA.

Pharmacokinetic Assessments: Whole blood samples of approximately 5 mL are collected for measurement of serum concentrations of tezepelumab.

Pharmacodynamic Assessments. Venous blood samples are collected from all subjects at the time points outlined to determine the effect of tezepelumab on the levels of circulating CCL17 (TARC) and CCL22 (MDC), which are downstream of TSLP signaling (Soumelis et al, 2002).

Pharmacogenetic Assessments. DNA analyses may be performed. These optional pharmacogenetic analyses focus on inherited genetic variations to evaluate their possible correlation to the disease and/or responsiveness to the therapies used in this study. The goals of the optional studies include the use of genetic markers to help in the investigation of CRS with or without nasal polyps and/or to identify subjects who may have positive or negative response to investigational product or protocol-required therapies.

Antibody Testing Procedures: Bioanalytical testing for anti-tezepelumab antibodies may be conducted if there are unexpected PK findings or safety-related concerns in the study population that warrant further investigation. Samples that test positive for anti-tezepelumab antibodies may be further characterized.

Blood Biomarkers: Blood samples (serum/plasma and whole blood) are collected as directed for the assessment of changes in circulating biomarker levels that are elevated in subjects with CRS. Additional assays which may be performed on blood samples include measuring changes in mRNA transcripts after tezepelumab treatment.

### Statistical Analysis

For the primary endpoints, the treatment effect will be tested using a logistic regression model adjusting for covariates. From this model, odd ratios and 95% Cls will be reported comparing each tezepelumab dose group to placebo. In addition, the percentage of subjects in each treatment group with a response and the difference in the percentage of subjects responding between each tezepelumab dose group and placebo will be summarized with a 95% confidence interval.

Approximately 400 adult participants with severe CRSwNP (total NPS ≥5) and an inadequate response to standard of care therapy are randomized to tezepelumab 210 mg Q4W or matching placebo in a 1:1 ratio.

Endpoints of interest: Change from baseline in co-primary endpoints NPS and bi-weekly mean NCS at Week 52 is measured and the difference in means between tezepelumab and placebo treatment groups used as efficacy measurement. Analyses of the key secondary endpoints are described as follows: the change from baseline in LMK, SNOT-22, loss of smell, and NPSD TSS at Week 52 is analyzed using the method as described in the primary analyses approach for the co-primary endpoints.

Time to first decision of surgery or SCS for NP during the 52-week treatment period is analyzed using a Cox proportional hazards model, adjusting for treatment, baseline co morbid asthma/AERD/NSAID-ERD status, prior surgery status, and regions. The event of interest is the first SCS use or time to decision for nasal polyp surgery, whichever is earlier if both occur. The date the decision was made to have the NP surgery will be used in the analysis instead of the actual date of the NP surgery analysis. If the decision date is missing, the actual date of the NP surgery will be used instead. Hazard ratios and corresponding 95% Cls and p-values will be presented. The proportion of participants having surgery or SCS up to week 52 will be estimated using the Kaplan-Meier method. The same method will be used to analyze the time to first surgery and time to first SCS separately.

The proportion of participants who achieve a maximum NPS≤1 in each nostril will be analyzed as follows: A participant who achieves a maximum score of 1 (NPS≤1) in each nostril at Week 52 in the absence of SCS or actual surgery at or prior to that time point will be defined as a responder, otherwise the participant will be defined as a non-responder. Participants who discontinued treatment prior to Week 52 will be considered non-responders. The response variable in the model will be the binary responder status at Week 52. A logistic regression model will be used, with treatment group, region, baseline co-morbid asthma/AERD/NSAID-ERD status, and prior surgery status as factors and baseline NPS as a covariate. Odds ratios, corresponding 95% Cls, and p-values will be presented. The same logistic regression model will also be used to analyze the proportion of participants who achieve a maximum NPS≤1 in each nostril and NPSD TSS response. The details for NPSD TSS response will be provided in the SAP.

Analysis of the change from baseline in FEV1 at Week 52 will also follow the method as described in the primary analysis approach for the co-primary endpoints; however, the population of interest will be the subset of participants with comorbid asthma/AERD/NSAID-ERD. The model includes baseline value of FEV1 as a covariate and prior surgery status, and regions as factors.

### Results

In an initial study (NAVIGATOR), patients (12-80 years old) were randomized 1:1 to tezepelumab 210 mg or placebo subcutaneously every 4 weeks for 52 weeks. Change from baseline to week 52 in SNOT-22 score (0 [no impairment] to 110 [maximum impairment]) was assessed in patients with any reported history of NP, grouped by whether NP was reported in the 2 years before randomization. Included patients had to have experienced at least two documented asthma exacerbations (in the 12 months before the date of informed consent) that led to hospitalization, an emergency department visit that required systemic corticosteroid treatment for at least 3 consecutive days, or the use of systemic corticosteroid treatment for at least 3 consecutive days.

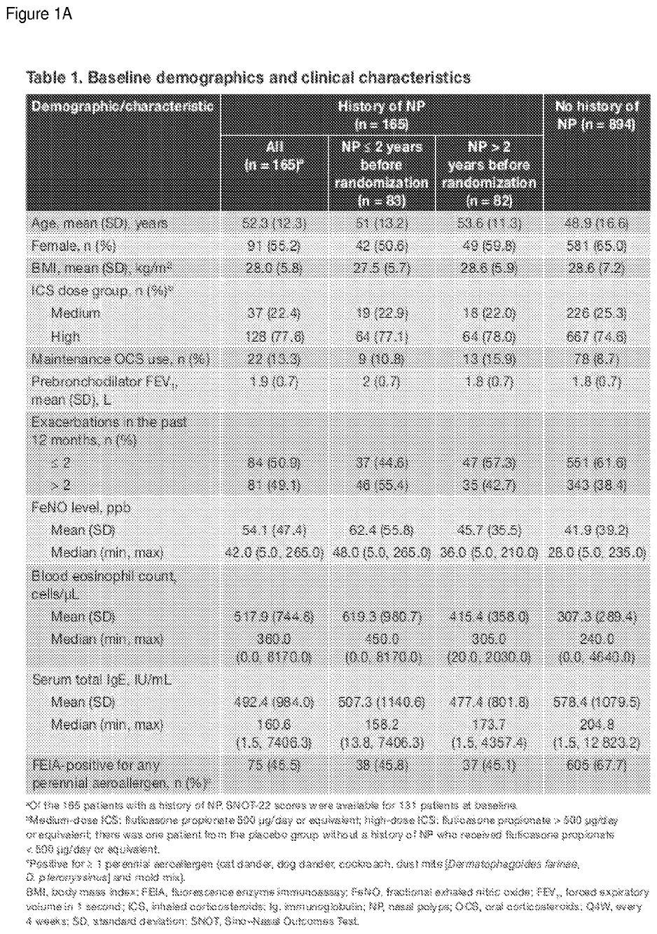

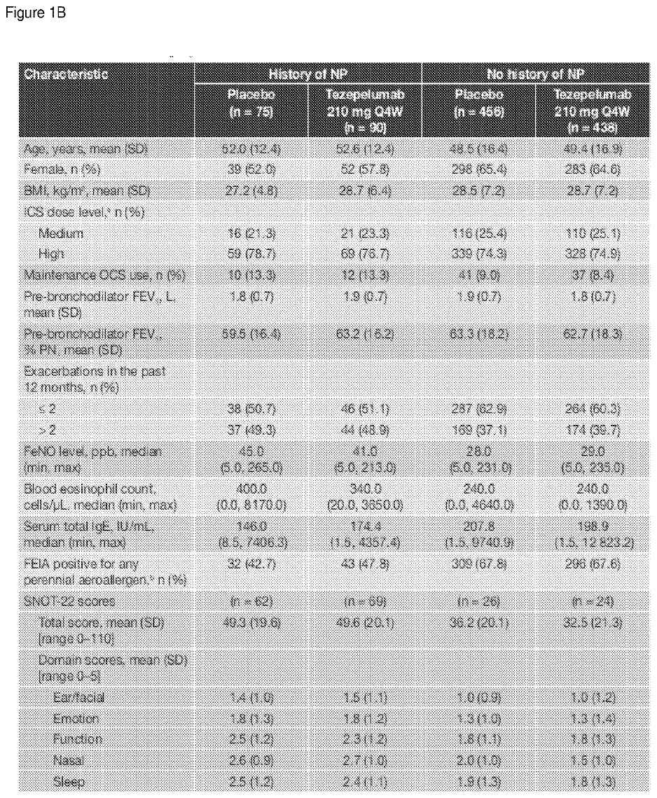

Baseline demographics and clinical characteristics were generally balanced between subgroups (FIG. 1A). Compared with patients with no history of NP, fewer patients with any history of NP were female (65.0% vs 55.2%) or were fluorescence enzyme immunoassay-positive for any perennial aeroallergen (67.7% vs 45.5%). Patients with any history of NP more commonly had more than two exacerbations in the past 12 months (49.1% vs 38.4%) and had higher mean fractional exhaled nitric oxide levels (47.4 ppb vs 39.2 ppb) and blood eosinophil counts at baseline (744.8 cells/μL vs 289.4 cells/μL) compared with patients with no history of NP.

A further analysis is set out in FIG. 1B showing SNOT score and domain scores for enrolled subject. Changes in SNOT-22 total scores (0 [no impairment] to 110 [maximum impairment]; minimal clinically important difference, 8.97) and domain scores were assessed from baseline to week 28 and week 52 in patients with a history of NP. Patients with a history of NP had higher SNOT-22 total and domain scores at baseline than those with no history of NP (FIG. 1B).

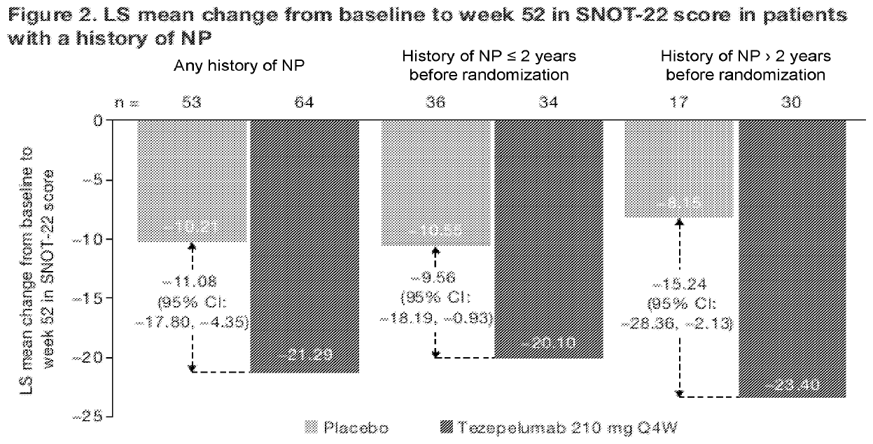

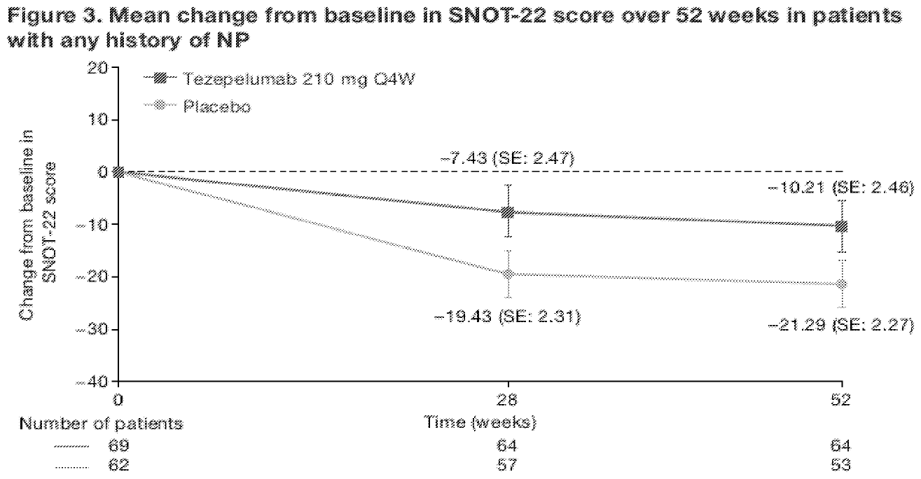

Mean baseline SNOT-22 scores were similar in patients receiving tezepelumab or placebo with any history of NP (49.6 [n=69] and 49.3 [n=62], respectively). In patients with any history of NP, change from baseline in SNOT-22 score was −21.29 for tezepelumab and −10.21 for placebo (LS mean difference, −11.08 [95% CI: −17.80, −4.35]). (FIG. 2). Of those, reductions from baseline in SNOT-22 score were observed in those without (tezepelumab, −23.40 [n=32]; placebo, −8.15 [n=22]; LS mean difference: −15.24 [95% CI: −28.36, −2.13]) and with NP in the 2 years before randomization (tezepelumab, −20.10 [n=37]; placebo, −10.55 [n=40]; LS mean difference: −9.56 [95% CI: −18.19, −0.93]). Clinically meaningful improvements in SNOT-22 score compared with placebo were observed from the first post-baseline timepoint assessment (mean change from baseline to week 28: tezepelumab, −19.43; placebo, −7.43) (FIG. 3).

When results were examined according to the chronology of NP history, reductions from baseline in SNOT-22 score were observed in those with NP in the 2 years before randomization and in those with NP more than 2 years before randomization (FIG. 2). These results show that tezepelumab provided clinically meaningful improvements in rhinosinusitis symptoms over 52 weeks versus placebo in patients with severe, uncontrolled asthma and remote or recent history of comorbid NP.

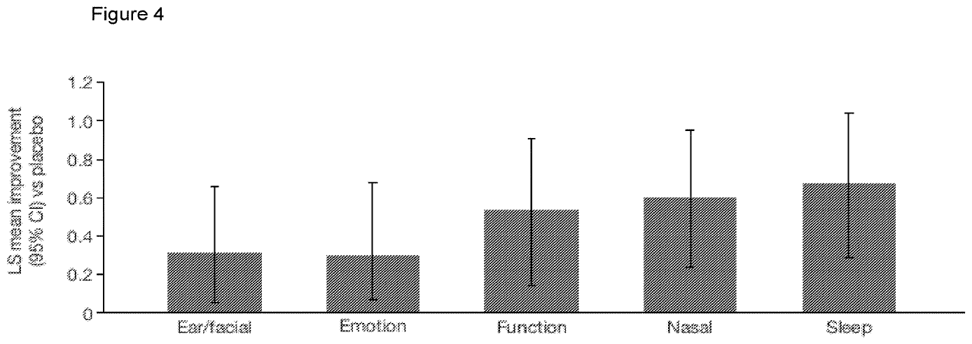

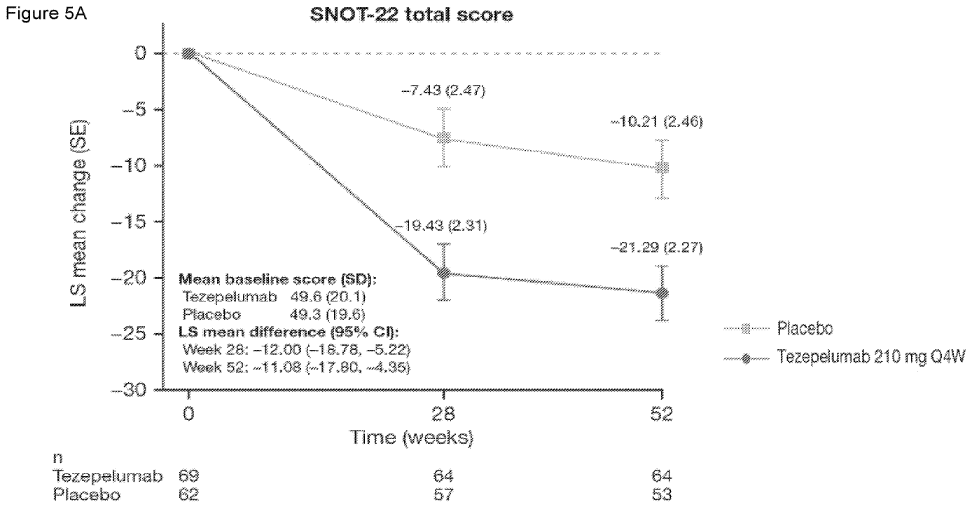

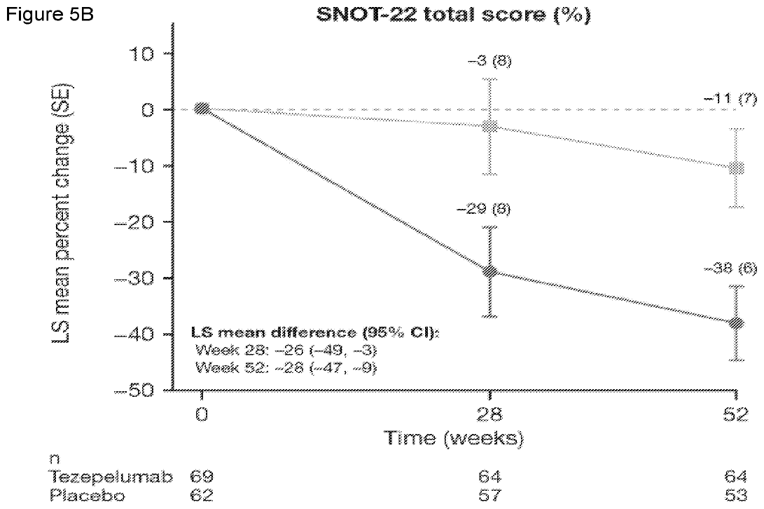

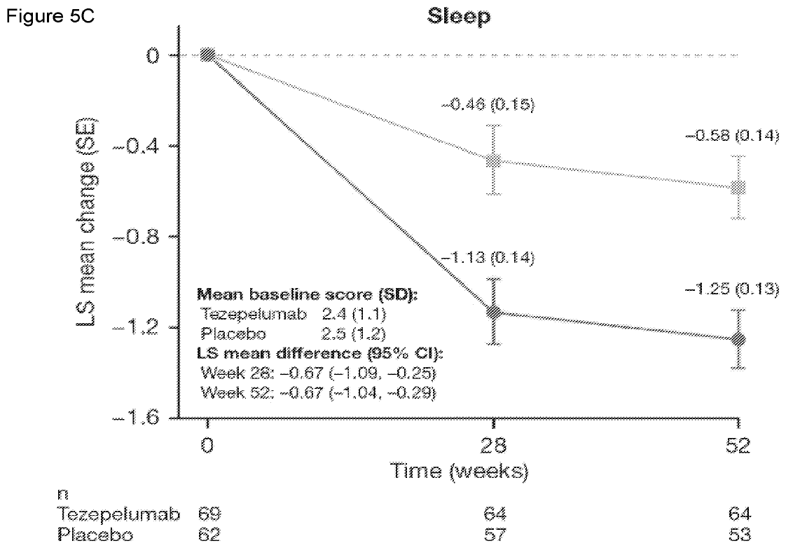

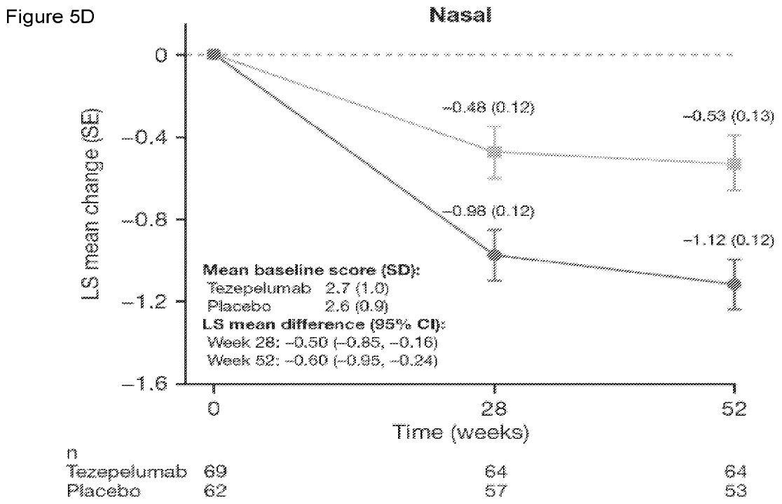

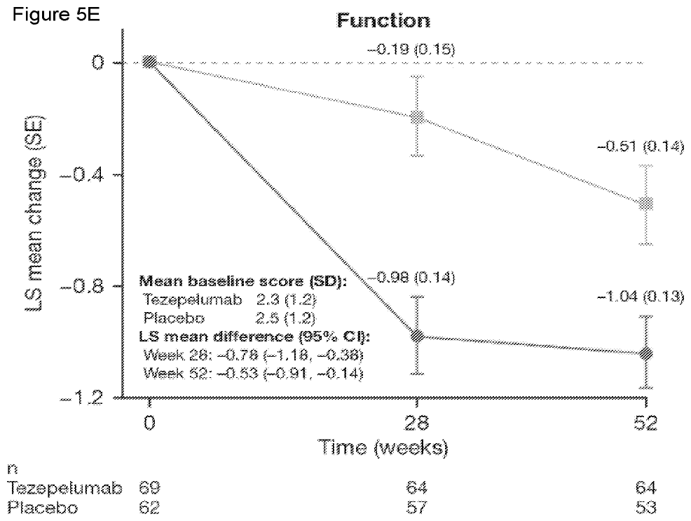

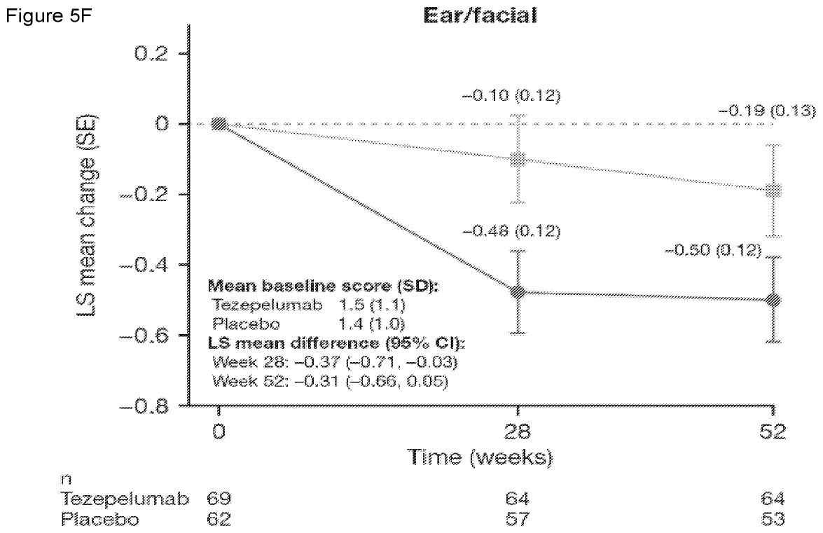

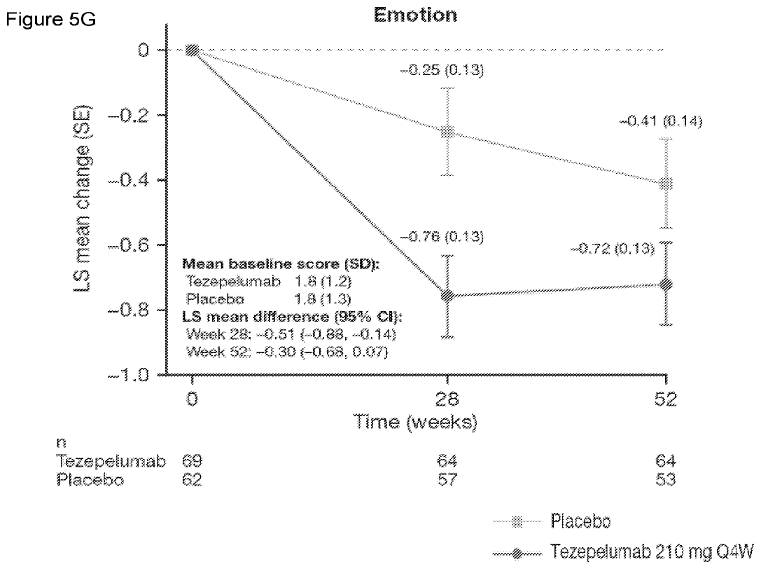

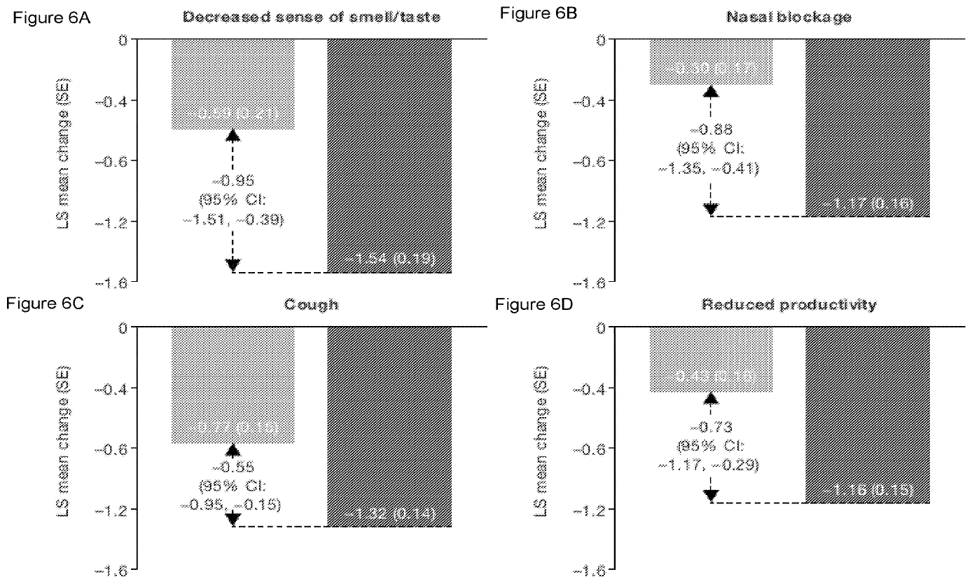

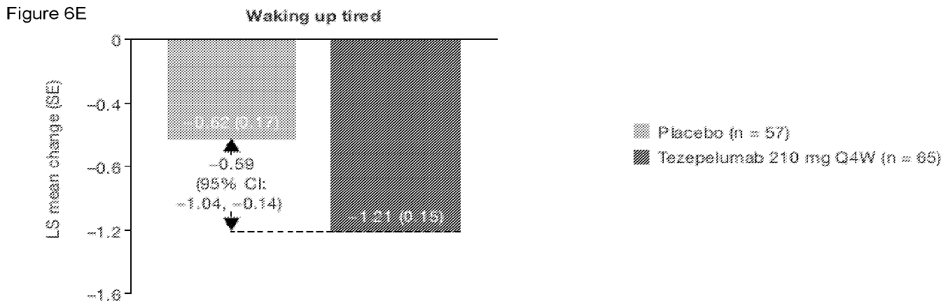

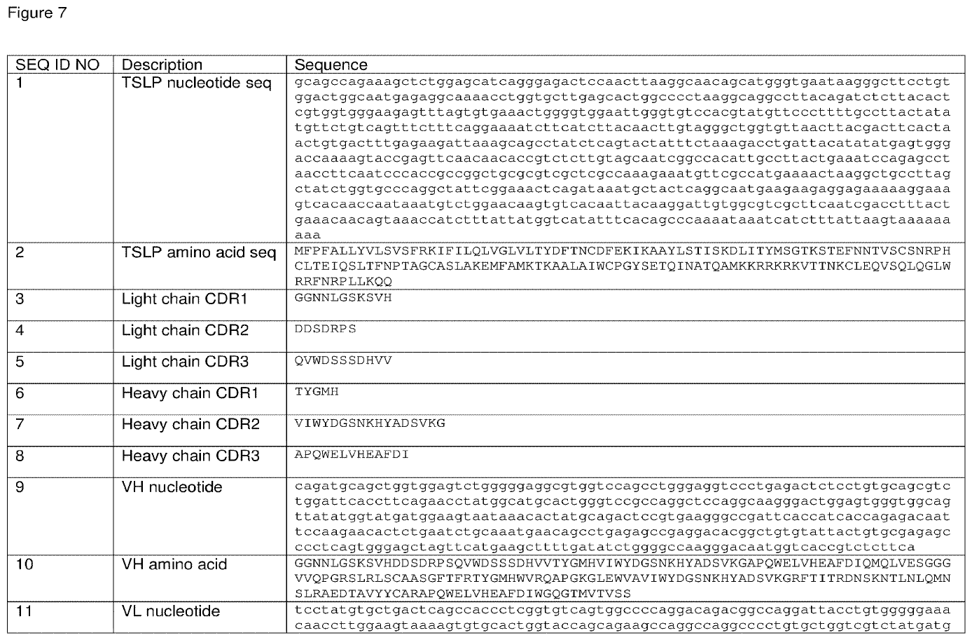

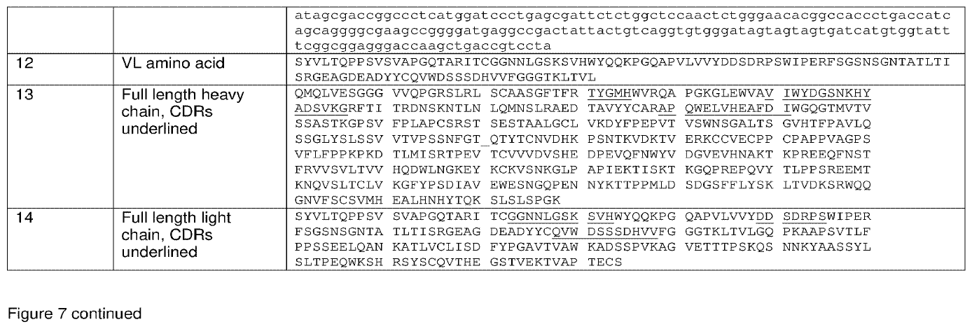

Tezepelumab numerically reduced each SNOT-22 domain score from baseline to week 28 and week 52 compared with placebo in patients with a history of NP (FIGS. 4 and 5). Percentage changes from baseline in SNOT-22 total scores were greater with tezepelumab than placebo both at week 28 (LS mean difference: −26% [95% confidence interval (CI): −49, −3]) and at week 52 (LS mean difference: −28% [95% CI: −47, −9]) (FIG. 5B). The SNOT-22 nasal domain was the most impaired at baseline, followed by the sleep, function, emotion and ear/facial domains (FIG. 5C-G). Tezepelumab reduced the following symptoms: decreased sense of smell/taste, nasal blockage, cough, reduced productivity and waking up tired, from baseline to week 52 compared with placebo. At week 52, the greatest improvements were seen in the sleep domain followed by the nasal and function domains. Tezepelumab reduced all five of the symptoms of most clinical interest. The greatest improvement was seen for the decreased sense of smell/taste, followed by nasal blockage, reduced productivity, waking up tired and cough (FIG. 6).

### Asthma-Related Clinical Outcomes

Tezepelumab reduced the AAER over 52 weeks compared with placebo by 69% (95% CI: 50, 81) in patients with a history of CRSwNP and by 53% (95% CI: 42, 62) in those without CRSwNP. Baseline pre-bronchodilator FEV1 (mean, standard deviation [SD]) was similar in patients with a history of CRSwNP (tezepelumab, 1.91 [0.72] L; placebo, 1.84 [0.71] L) and in those without CRSwNP (tezepelumab, 1.81 [0.72] L; placebo, 1.85 [0.71] L). Tezepelumab treatment resulted in an improvement in pre-bronchodilator FEV1 compared with placebo in patients regardless of their CRSwNP history. The LS mean difference (95% CI) for the change in pre-bronchodilator FEV1 from baseline to week 52 was 0.20 (0.07, 0.32) L in patients with a history of CRSwNP and 0.12 (0.07, 0.17) L in patients without.

Baseline ACQ-6 scores (mean [SD]) were similar in patients with a history of CRSwNP (tezepelumab, 2.86 [0.82]; placebo, 2.83 [0.86]) and in those without (tezepelumab, 2.81 [0.81]; placebo, 2.79 [0.81]). Tezepelumab treatment resulted in an improvement in ACQ-6 score compared with placebo. The LS mean difference (95% CI) for the change in ACQ-6 score from baseline to week 52 was −0.63 (−0.95, −0.31) in patients with a history of CRSwNP and −0.27 (−0.41, −0.13) in those without. Baseline AQLQ(S)+12 scores (mean [SD]) were similar in patients with a history of CRSwNP (tezepelumab, 3.90 [0.99]; placebo, 3.84 [1.01]) and in those without (tezepelumab, 3.86 [1.03]; placebo, 3.92 [1.00]). At week 52, the improvement in AQLQ(S)+12 score with tezepelumab compared with placebo was greater in patients with a history of CRSwNP (LS mean difference [95% CI]: 0.77 [0.43, 1.12] than in those without (LS mean difference [95% CI]: 0.25 [0.10, 0.40]). Baseline ASD scores (mean [SD]) were similar in patients with a history of CRSwNP (tezepelumab, 1.41 [0.68]; placebo, 1.44 [0.71]) and in those without CRSwNP (tezepelumab, 1.39 [0.70]; placebo, 1.39 [0.69]). Tezepelumab improved ASD scores compared with placebo in patients with and without a history of CRSwNP; the LS mean difference (95% CI) for the change from baseline to week 52 was −0.32 (−0.51, −0.13) and −0.08 (−0.16, 0.01), respectively.

These results show numerical reductions were observed across all SNOT-22 domains, with the greatest reductions seen in the sleep, nasal and function domains. Tezepelumab improved the following symptoms: decreased sense of smell/taste, nasal blockage, cough, reduced productivity and waking up tired, over 52 weeks compared with placebo. These results demonstrate the efficacy of tezepelumab in improving rhinosinusitis symptoms in patients with severe, uncontrolled asthma and a history of NP.

Numerous modifications and variations of the invention as set forth in the above illustrative examples are expected to occur to those skilled in the art. Consequently only such limitations as appear in the appended claims should be placed on the invention.

### REFERENCES

Alobid et al., Allergy. 2005; 60:452-458.
Alving et al., Pulm Ther. 2017 3 (1): 207-218.
Agarwal et al., Clin Rev Allergy Immunol 2019; doi 10.1007/s12016-019-08734-z: May 10 [Epub ahead of print].
Bachert et al., JAMA. 2016; 315 (5): 469-79.
Bachert et al., Journal of Allergy and Clinical Immunology (2017), doi: 10.1016/j.jaci.2017.05.044.
Bachert et al., Lancet 2019. Available from: URL: https://doi.org/10.1016/S0140-6736 (19) 31881-1.
Bilodeau et al., Rhinology. 2010; 48:420-425.
Chaaban et al., Am J Rhinol Allergy 2013; 27:473-478.
Chitsuthipakorn W et al., Curr Allergy Asthma Rep. 2018; 18 (9): 46.
Deal and Kountakis et al., Laryngoscope 2004; 114:1932-1935.
Doty et al., Physiology & Behavior (Monograph) 1984 32:489-502.
Ediger et al., Clinical & Experimental Allergy, 35 (3), 319-326.
Ehnhage A et al., Allergy. 2009; 64:762-769.
Emson C et al., Am J Respir Crit Care Med 2020; 201: A4256.
Emson et al., Presented at: European Academy of Allergy and Clinical
Immunology (EAACI) Digital Congress 2020.
Emson et al., J Asthma Allergy. 2021; 14:91-99.
Fokkens et al., Rhinol Suppl 2020 58 Supplement S29 (1-464).
Gauvreau et al., Expert opinion on therapeutic targets, 1-16.
Gevaert et al., J Allergy Clin Immunol 2011; 128:989-95.
Gevaert et al., Journal of Allergy and Clinical Immunology 2020.
Graham et al., Am J Respir Crit Care Med. 2019; 200 (8): e70-e88.
Håkansson et al., PLOS ONE 2015; 10 (7): e0127228.
Hopkins et al., Clinical otolaryngology 2009; 34 (5): 447-5.
Hull and Chandra Otolaryngol Clin North Am 2017; 50 (1): 61-81.
Jarvis et al., Allergy. 2012; 67:91-98.
Juniper et al., Respir Med 2006; 100:616-621.
Kato et al., Journal of Allergy and Clinical Immunology, 143 (2), AB86.
Kimura et al., Allergy Asthma Immunol Res. 2011; 3 (3): 186-193.
Lieberman et al., J Allergy Clin Immunol. 2010 September; 126 (3): 477-80. e1-42.
Likness et al., Otolaryngol Head Neck Surg. 2014 February; 150 (2): 305-311.
Lou et al., Int Forum Allergy Rhinol. 2018 November; 8 (11): 1218-1225.
Lund and Mackay, Rhinology 1993; 31 (4): 183-4.
Nagarkar et al. J Allergy Clin Immunol. 2013; 132 (3): 593-600 e512.
Okushi et al., Auris Nasus Larynx 40 (2013) 548-553.
Orlandi et al., Int Forum Allergy Rhinol 2016; 6 (Suppl. 1): S22-S209.
Pallanch et al., Int Forum Allergy Rhinol 2013; 3 (12): 963-72.
Pearlman et al., Am J Rhinol Allergy. 2009; 23:145-148.
Piccirillo et al., Otolaryngology—Head and Neck Surgery. 2002; 126 (1); 41-7.
Quanjer et al. Multi ethnic reference values for spirometry for the 3-95 year age range: the global lung function 2012 equations. 2012 December; 40 (6): 1324-43.
QualityMetric, I. User's manual for the SF-36v2 Health Survey (3 ed.). 2011.
Lincoln, RI.
Reilly et al., PharmacoEconomics 1993; 4 (5): 353-365.
Sampson et al., J Allergy Clin Immunol. 2006 February; 117 (2): 391-7.
Scadding et al., Clin Transl Allergy. 2011; 1:1-39.
Settipane et al., J Allergy Clin Immunol. 1977; 59:17-21.
Seybt et al., Ear Nose Throat J 2007; 86:409-11.
Shin et al., American journal of rhinology & allergy, 28 (2), 95-98.
Stevens et al., Clin Immunol Pract 2016; 4 (4): 565-72.
Tint et al., Curr Allergy Asthma Rep 2016; 16:16.
Tokunaga et al., Allergy 2015; 70:995-1003.
van der Veen et al. Allergy 2017; 72:282-290.

What is claimed:

1. A method for treating chronic rhinosinusitis in a subject comprising selecting a subject in need of treatment for chronic rhinosinusitis, and administering a therapeutically effective amount of an anti-TSLP antibody or antibody variant to the subject, wherein the antibody comprises
a. a light chain variable domain comprising:
i. a light chain CDR1 sequence comprising the amino acid sequence set forth in SEQ ID NO: 3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

2. A method for treating chronic rhinosinusitis in a subject comprising selecting a subject in need of treatment for chronic rhinosinusitis, and administering a therapeutically effective amount of an anti-TSLP antibody or antibody variant to the subject, wherein the antibody comprises
a. a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO: 11; and
b. a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b), wherein the antibody specifically binds to a TSLP polypeptide as set forth in amino acids 29-159 of SEQ ID NO:2.

3. The method of claim 1 or 2, wherein the antibody is administered every 2 weeks or every 4 weeks.

4. The method of any one of claims 1 to 3, wherein the antibody is an IgG2 antibody.

5. The method of any one of claims 1 to 4, wherein the antibody is administered at a dose of between 140 and 420 mg.

6. The method of any one of claims 1 to 5, wherein the antibody is administered at a dose of 210 mg.

7. The method of any one of claims 1 to 6, wherein the antibody is administered at a dose of 420 mg.

8. A method for treating chronic rhinosinusitis in a subject comprising selecting a subject in need of treatment for chronic rhinosinusitis, and administering a composition comprising an anti-TSLP antibody to the subject in a dose of 210 mg at an interval of every 4 weeks, wherein the antibody comprises
a. a light chain variable domain comprising:
i. a light chain CDR1 sequence comprising the amino acid sequence set forth in SEQ ID NO: 3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

9. A method for treating chronic rhinosinusitis in a subject comprising selecting a subject in need of treatment for chronic rhinosinusitis, and administering a composition comprising an anti-TSLP antibody to the subject in a dose of 210 mg at an interval of every 4 weeks, wherein the antibody comprises
a. a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO: 11; and
b. a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b), wherein the antibody specifically binds to a TSLP polypeptide as set forth in amino acids 29-159 of SEQ ID NO:2.

10. The method of any one of the preceding claims wherein the light chain variable domain is set out in SEQ ID NO: 12 and the heavy chain variable domain is set out in SEQ ID NO:10.

11. The method of any one of the preceding claims, wherein the antibody is administered for a period of at least 4 months, 6 months, 9 months, 1 year or more.

12. The method of any one of the preceding claims, wherein said anti-TSLP antibody is selected from the group consisting of a monoclonal antibody, a recombinant antibody, an IgG1 antibody, an IgG2 antibody, an IgG3 antibody, and an IgG4 antibody.

13. The method of any one of claims 9-12, wherein the antibody is an IgG2 antibody.

14. The method of any one of the preceding claims, wherein the antibody is a human antibody.

15. The method of any one of the preceding claims, comprising administering to a patient a pharmaceutical composition comprising the antibody or antibody variant and further comprising a pharmaceutically acceptable carrier or excipient.

16. The method of any one of the preceding claims, wherein the chronic rhinosinusitis is severe or moderate chronic rhinosinusitis.

17. The method of any one of the preceding claims, wherein the chronic rhinosinusitis is with nasal polyps.

18. The method of any one of the preceding claims, wherein the subject has moderate asthma.

19. The method of any one of the preceding claims, wherein the subject is an adult.

20. The method any one of the preceding claims, wherein the subject is a child or adolescent.

21. The method any one of the preceding claims, wherein the administration improves one or more measures of chronic rhinosinusitis including Nasal Polyp Score (NSP), Nasal Congestion Score (NCS), loss of smell, SinoNasal Outcome Test 22 Item (SNOT-22), EuroQOL quality of life 5-dimensions 3-level version (EQ-5D-3L), Asthma Control Questionnaire (ACQ-6), Lund-Mackay score, modified Lund-Mackay Score, incidence of nasal polyp surgery and/or systemic corticosteroids (SCS).

22. The method any one of the preceding claims, wherein the administration improves one or more symptoms of chronic rhinosinusitis as measured by a patient symptom diary.

23. The method of any one of the preceding claims, wherein the administration improves one or more symptoms of chronic rhinosinusitis selected from the group consisting of nasal blockage, nasal congestion, runny nose, post-nasal drip, mucus drainage down the throat, prominent nasal obstruction, nasal discharge, loss of smell, asthma symptoms, headache, facial pain, facial pressure, difficulty with sense of smell, difficulty with sleeping due to nasal symptoms, difficulty with daily activities due to nasal symptoms.

24. A method for treating chronic rhinosinusitis in a subject comprising selecting a subject in need of treatment for chronic rhinosinusitis, and administering a composition comprising an anti-TSLP antibody to the subject in a dose of 140 to 420 mg at an interval of every 2 weeks, wherein the antibody comprises
a. a light chain variable domain comprising:
i. a light chain CDR1 sequence comprising the amino acid sequence set forth in SEQ ID NO: 3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2, wherein the antibody is an IgG2 antibody.

25. The method of claim 24, wherein the light chain variable domain is set out in SEQ ID NO: 12 and the heavy chain variable domain is set out in SEQ ID NO: 10.

26. The method of claim 24, wherein the antibody is administered every 4 weeks.

27. The method of claim 24 or 25, wherein the antibody is administered at a dose of 210 mg.

28. The method of any one of claims 24 to 27, wherein the antibody is administered at a dose of 420 mg.

29. The method of any one of the preceding claims, wherein the antibody is tezepelumab.

30. The method of claim 29, wherein the antibody is an IgG2 antibody, and has the full length heavy and light chain sequences set out in SEQ ID NOs: 13 and 14, respectively.

31. The method of any one of the preceding claims, wherein the antibody variant has substantially similar pK characteristics as tezepelumab in humans.

32. A method of reducing the frequency of chronic rhinosinusitis exacerbation in a subject comprising selecting a subject in need of treatment for chronic rhinosinusitis, and administering a composition comprising an anti-TSLP antibody to the subject in a dose of 140 mg to 420 mg at an interval of every 2 weeks or every 4 weeks, wherein the antibody comprises
a. a light chain variable domain comprising:
i. a light chain CDR1 sequence comprising the amino acid sequence set forth in SEQ ID NO: 3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

33. A method of reducing the frequency of chronic rhinosinusitis exacerbation in a subject comprising selecting a subject in need of treatment for chronic rhinosinusitis, and administering a composition comprising an anti-TSLP antibody to the subject in a dose of 140 mg to 420 mg at an interval of every 2 weeks or every 4 weeks, wherein the antibody comprises
a. a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO: 11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b).

34. The method of claim 32 or 33, wherein the light chain variable domain is set out in SEQ ID NO: 12 and the heavy chain variable domain is set out in SEQ ID NO: 10.

35. The method of any one of claims 32 to 34, wherein the antibody is administered every 4 weeks.

36. The method of any one of claims 32 to 34, wherein the antibody s administered at a dose of 210 mg.

37. The method of any one of claims 32 to 34, wherein the s administered at a dose of 420 mg.

38. The method of any one of claims 32 to 37, wherein the antibody is administered for a period of at least 4 months, 6 months, 9 months, 1 year or more.

39. The method of any one of claims 32 to 38, wherein said anti-TSLP antibody is selected from the group consisting of a monoclonal antibody, a recombinant antibody, an IgG1 antibody, an IgG2 antibody, an IgG3 antibody, and an IgG4 antibody.

40. The method any one of claims 32 to 39, wherein the antibody is an IgG2 antibody.

41. The method of any one of claims 32 to 40, wherein the is a human antibody.

42. The method of any one of claims 32 to 41 wherein the antibody is tezepelumab.

43. The method of any one of claims 32 to 42, comprising administering to a patient a pharmaceutical composition comprising the antibody or antibody variant and further comprising a pharmaceutically acceptable carrier or excipient.

44. The method of any one of claims 32 to 43, wherein the administration delays the time to a chronic rhinosinusitis exacerbation compared to a subject not receiving the anti-TSLP antibody.

45. The method any one of claims 32 to 45, wherein the administration reduces frequency of or levels of co-administered therapy in the subject.

46. The method of claim 45, wherein the co-administered therapy is selected from the group consisting of dupilumab, immunosuppressive or immunomodulating drugs, systemic corticosteroids, cyclosporine, mycophenolate-mofetil, interferon (IFN)-gamma, Janus kinase inhibitors, azathioprine, methotrexate, anti-IL-13 antibodies, anti-IL-5 pathway antibodies, or combinations thereof.

47. The method of claim 45, wherein the administration reduces or eliminates the need for corticosteroid therapy.

48. A method for reducing SNOT-22 and/or Lund-Mackay score in a subject comprising administering a composition comprising of an anti-TSLP antibody in a dose of 140 mg to 420 mg at an interval of every 2 weeks, wherein the antibody comprises
a. a light chain variable domain comprising:
i. a light chain CDR1 sequence comprising the amino acid sequence set forth in SEQ ID NO: 3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

49. A method for reducing SNOT-22 and/or Lund-Mackay score in a subject comprising administering a composition comprising an anti-TSLP antibody in a dose of 140 mg to 420 mg at an interval of every 2 weeks, wherein the antibody comprises
a. a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO: 11; and
b. a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b).

50. A method for reducing SNOT-22 and/or Lund-Mackay score in a subject comprising administering a composition comprising an anti-TSLP antibody in a dose of 210 mg at an interval of every 4 weeks, wherein the antibody comprises
a. a light chain variable domain comprising:
i. a light chain CDR1 sequence comprising the amino acid sequence set forth in SEQ ID NO: 3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

51. A method for reducing SNOT-22 and/or Lund-Mackay score in a subject comprising administering a composition comprising an anti-TSLP antibody in a dose of 210 mg at an interval of every 4 weeks, wherein the antibody comprises
a. a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO: 11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b).

52. The method of any one of claims 48 to 51, wherein the light chain variable domain is set out in SEQ ID NO: 12 and the heavy chain variable domain is set out in SEQ ID NO: 10.

53. The method of any one of claims 48 to 52 wherein the subject has a nasal polyp score (NPS) of 0 or 1 after 52 weeks of treatment.

54. The method of any one of claims 32 to 53, wherein the anti-TSLP antibody is tezepelumab.

55. The method of claim 54, wherein the antibody is an IgG2 antibody, and has the full length heavy and light chain sequences set out in SEQ ID NOs: 13 and 14, respectively.

56. The method of any one of claims 48-55, wherein the antibody is administered every 4 weeks.

57. The method of any one of claims 24 to 56, wherein the subject has chronic rhinosinusitis.

58. The method of claim 57, wherein the chronic rhinosinusitis is severe or moderate chronic rhinosinusitis.

59. The method any one of the preceding claims, wherein the administration is subcutaneous or intravenous.

60. A method for treating chronic rhinosinusitis with nasal polyps in a subject comprising selecting a subject in need of treatment for chronic rhinosinusitis with nasal polyps, and administering a composition comprising an anti-TSLP antibody to the subject in a dose of 210 mg at an interval of every 4 weeks, wherein the antibody comprises
a. a light chain variable domain comprising:
i. a light chain CDR1 sequence comprising the amino acid sequence set forth in SEQ ID NO: 3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8;

61. A method for treating chronic rhinosinusitis with nasal polyps in a subject comprising selecting a subject in need of treatment for chronic rhinosinusitis with nasal polyps, and administering a composition comprising an anti-TSLP antibody to the subject in a dose of 210 mg at an interval of every 4 weeks, wherein the antibody comprises
a. a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO: 11;
iii. a sequence of amino acids encoded by a polynucleotide that hybridizes under moderately stringent conditions to the complement of a polynucleotide consisting of SEQ ID NO: 11; and
b. a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO: 9; or
c. a light chain variable domain of (a) and a heavy chain variable domain of (b),
wherein the antibody specifically binds to a TSLP polypeptide as set forth in amino acids 29-159 of SEQ ID NO:2.

62. The method of claim 60 or 61, wherein the light chain variable domain is set out in SEQ ID NO: 12 and the heavy chain variable domain is set out in SEQ ID NO: 10.

63. The method of claim 62, wherein the antibody is an IgG2 antibody, and has the full length heavy and light chain sequences set out in SEQ ID NOs: 13 and 14, respectively.

64. The method of any one of claims 60 to 63, wherein the antibody or antibody variant is administered for a period of at least 4 months, 6 months, 9 months, 1 year or more.

65. The method of any one of claims 60 to 64, wherein said anti-TSLP antibody is selected from the group consisting of a monoclonal antibody, a recombinant antibody, an IgG1 antibody, an IgG2 antibody, an IgG3 antibody, and an IgG4 antibody.

66. The method any one of claims 60 to 65, wherein the antibody is an IgG2 antibody.

67. The method of any one of claims 60 to 66, wherein the antibody is a human antibody.

68. The method of any one of claims 60 to 67 wherein the antibody is tezepelumab.

69. A method for treating chronic rhinosinusitis in a subject comprising selecting a subject in need of treatment for chronic rhinosinusitis, and administering a therapeutically effective amount of an anti-TSLP antibody or antibody variant to the subject, wherein the anti-TSLP antibody or antibody variant is described in WO 20220226342A1, WO 20220226339A1, WO2023098491A1, WO2021155634A1, WO2022166072A1, WO2021043221A1, WO2022184074A1, WO2021104053A1, WO2023116925A1, WO2021155861A1, WO2022116858A1, WO2022117079A1, WO2020244544A1, WO2021152488A1, WO2022253147A1, WO2023070948A1, WO2023142309A1, WO2022095689A1, WO2021115240A1, WO2022166739A1 and WO2019100111A1 or set out in Table A.

70. The method of any one of claims 60 to 69, comprising administering to a patient a pharmaceutical composition comprising the antibody or antibody variant and further comprising a pharmaceutically acceptable carrier or excipient.

71. The method of any one of claims 60 to 70, wherein the administration delays the time to a chronic rhinosinusitis exacerbation compared to a subject not receiving the anti-TSLP antibody.

72. The method any one of claims 60 to 71, wherein the administration reduces frequency of or levels of co-administered therapy in the subject.

73. The method of claim 72, wherein the co-administered therapy is selected from the group consisting of dupilumab, immunosuppressive or immunomodulating drugs, systemic corticosteroids, cyclosporine, mycophenolate-mofetil, interferon (IFN)-gamma, Janus kinase inhibitors, azathioprine, methotrexate, anti-IL-13 antibodies, anti-IL-5 pathway antibodies, or combinations thereof.

74. The method of claim 72 or 73, wherein the administration reduces or eliminates the need for corticosteroid therapy.

75. The method of any one of claims 60 to 74 wherein the subject has a nasal polyp score (NPS) of 0 or 1 after 52 weeks of treatment.

76. The method any one of claims 60 to 75, wherein the administration improves one or more measures of chronic rhinosinusitis including Nasal Polyp Score (NSP), Nasal Congestion Score (NCS), loss of smell, SinoNasal Outcome Test 22 Item (SNOT-22), EuroQOL quality of life 5-dimensions 3-level version (EQ-5D-3L), Asthma Control Questionnaire (ACQ-6), Lund-Mackay score, modified Lund-Mackay Score, incidence of nasal polyp surgery and/or systemic corticosteroids (SCS).

77. The method any one of claims 60 to 76, wherein the administration improves one or more symptoms of chronic rhinosinusitis as measured by a patient symptom diary.

78. The method of any one of claims 60 to 77, wherein the administration improves one or more symptoms of chronic rhinosinusitis selected from the group consisting of nasal blockage, nasal congestion, runny nose, post-nasal drip, mucus drainage down the throat, prominent nasal obstruction, nasal discharge, loss of smell, asthma symptoms, headache, facial pain, facial pressure, difficulty with sense of smell, difficulty with sleeping due to nasal symptoms, difficulty with daily activities due to nasal symptoms.

79. The method of any one of claims 60 to 78, wherein the chronic rhinosinusitis. is severe or moderate chronic rhinosinusitis.

80. The method any one of claims 60 to 79, wherein the administration is subcutaneous or intravenous.


SEQUENCE LISTING (14 sequences)

SEQ ID NO: 1 - DNA, 743 bp, Homo sapiens
  source 1..743: mol_type = genomic DNA
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, synthetic construct
GGNNLGSKSVH

SEQ ID NO: 4 - protein, 7 aa, synthetic construct
DDSDRPS

SEQ ID NO: 5 - protein, 11 aa, synthetic construct
QVWDSSSDHVV

SEQ ID NO: 6 - protein, 5 aa, synthetic construct
TYGMH

SEQ ID NO: 7 - protein, 17 aa, synthetic construct
VIWYDGSNKHYADSVKG

SEQ ID NO: 8 - protein, 13 aa, synthetic construct
APQWELVHEAFDI

SEQ ID NO: 9 - DNA, 366 bp, synthetic construct
  source 1..366: mol_type = other DNA
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 186 aa, synthetic construct
GGNNLGSKSVHDDSDRPSQVWDSSSDHVVTYGMHVIWYDGSNKHYADSVKGAPQWELVHEAFDIQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, synthetic construct
  source 1..325: mol_type = other DNA
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 448 aa, Homo sapiens
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 214 aa, Homo sapiens
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS